SEQUENCE LISTING (20 sequences)

SEQ ID NO: 1 - DNA, 31 bp, Artificial
  primer
ttaaaaggattgttttatatatttaaggaat

SEQ ID NO: 2 - DNA, 28 bp, Artificial
  primer
cactcactttattcaacatctaaaaaac

SEQ ID NO: 3 - DNA, 18 bp, Artificial
  primer
atttaggtgacactatag

SEQ ID NO: 4 - DNA, 20 bp, Artificial
  primer
taatacgactcactataggg

SEQ ID NO: 5 - DNA, 21 bp, Artificial
  NRSF/REST
gcuuauuaugcuggcaaautt

SEQ ID NO: 6 - DNA, 21 bp, Artificial
  NRSF/REST
gccuucuaauaaugugucatt

SEQ ID NO: 7 - DNA, 25 bp, Artificial
  probe
aggaaggccgaatacagttatggcc

SEQ ID NO: 8 - DNA, 25 bp, Artificial
  probe
cctgctgaaggacgctgccaaggtc

SEQ ID NO: 9 - DNA, 25 bp, Artificial
  probe
cgagtaccccttcaggctgcaccaa

SEQ ID NO: 10 - DNA, 25 bp, Artificial
  probe
gactgaacagtcaccgacgagagtg

SEQ ID NO: 11 - DNA, 20 bp, Artificial
  primer
acaaatcccgtctccacaac

SEQ ID NO: 12 - DNA, 21 bp, Artificial
  primer
gcctagggaagacgaaaaaca

SEQ ID NO: 13 - DNA, 20 bp, Artificial
  primer
gctccgtagctgtttttcgt

SEQ ID NO: 14 - DNA, 20 bp, Artificial
  primer
gccactcactttgttcagca

SEQ ID NO: 15 - DNA, 17 bp, Artificial
  consensus
ntyagmrccnnrgmsag

SEQ ID NO: 16 - DNA, 21 bp, Homo sapiens
gtgcgcagcgcggagtggtac

SEQ ID NO: 17 - DNA, 21 bp, Homo sapiens
caccgctacccggagagcgcg

SEQ ID NO: 18 - DNA, 21 bp, Homo sapiens
atcagcgactcggctccccct

SEQ ID NO: 19 - DNA, 84 bp, Artificial
  consensus
cagattatctcaccggcgagtgagactgcaaggtttgggggcccggccgtaccactccgcgctgcgcacggggggttcgtaccc

SEQ ID NO: 20 - DNA, 84 bp, Artificial
  PCR product
tagattattttatcggtgagtgagattgtaaggtttgggggtttggttgtattattttgtgttgcgtatggggggtttgtattt

(19) United States
(12) Patent Application Publication
(10) Pub. No.: US 2010/0247543 A1
(43) Pub. Date: Sep. 30, 2010

(54) TREATMENT AND PREVENTION OF NEURODEGENERATIVE DISEASES

(75) Inventors: Tamara Maes, Barcelona (ES); Marta Barrachina Castillo, Barcelona (ES); Isidro Ferrer Abizanda, Barcelona (ES)

Correspondence Address: Workman Nydegger;1000 Eagle Gate Tower, 60 East South Temple, Salt Lake City, UT, 84111, US

(73) Assignee: ORYZON GENOMICS, S.A., BARCELONA (ES)

(21) Appl. No.: 12/711,938

(22) Filed: Feb. 24, 2010

Related U.S. Application Data
(63) Continuation of application No. PCT/EP2008/061036, filed on Aug. 22, 2008.

(30) Foreign Application Priority Data
Aug. 24, 2007 (GB) 0716577.2
Oct. 11, 2007 (GB) 0719899.7
Dec. 21, 2007 (GB) 0724970.9

(51) Int. Cl. A61K 39/395; A61P 25/28; A61P 25/00; A61K 31/16; A61K 31/4436; A61K 31/045; A61K 31/19; A61K 31/13; A61K 31/70; A61K 31/7004; A61K 38/44; A61K 31/135; A61K 31/7088; G01N 33/53

(52) U.S. Cl. 424/139.1; 514/575; 514/336; 514/724; 514/557; 514/674; 514/62; 514/23; 424/94.4; 514/647; 514/44 R; 514/44 A; 435/7.2

(57) ABSTRACT

The treatment and prevention of neurodegenerative diseases by repression of the transcriptional complex that silences the promoter of the UCHL1 gene.

### CROSS-REFERENCE TO RELATED APPLICATIONS

This application claims the benefit of PCT/EP2008/061036, filed on Aug. 22, 2008 and designating the United States, which claims the benefit of Great Britain patent application Nos., 0716577.1, filed Aug. 24, 2007, 0719899.7, filed Oct. 11, 2007, and 0724970.9, filed Dec. 21, 2007, all of which are hereby incorporated herein by reference in their entirety.

### BACKGROUND OF THE INVENTION

1. The Field of the Invention

Neurodegenerative diseases are conditions in which cells of the brain and spinal cord are lost. Probably the best known neurodegenerative diseases are Alzheimer's disease, Parkinson's disease and multiple sclerosis which are caused by the gradual deterioration of neurons causing symptoms affecting cognition and/or movement, eventually leading to death.

“Lewy body disorders” is an umbrella term which includes dementia with Lewy bodies (DLB), Parkinson's disease (PD) and PD with dementia (PDD). These disorders are characterised by disorders of alpha-synuclein metabolism, which gives rise to the formation of abnormal neuronal alpha-synuclein inclusions, which are the defining pathologic process common to both PDD and DLB.

Synucleinopathies, with and without dementia, encompass a wide range of diseases including Parkinson's disease, multiple system atrophy, rapid eye movement (REM), sleep behaviour disorder, and dementia with Lewy bodies (DLB). DLB is a neurodegenerative disorder resulting in slowly progressive and unrelenting dementia until death. Prevalence studies suggest that it is the second most common dementing illness in the elderly. The neuropathologic findings of DLB show a wide anatomic range. Lewy bodies and Lewy-related pathology are found from the brain stem to the cortex and, in many cases, associated with concurrent Alzheimer's disease pathology.

PDD and DLB show differing temporal sequences of key symptoms and clinical features. Patients with Parkinson's disease show an increased risk for dementia based on epidemiological studies. The criteria by McKeith et al. (2005) (Neurology, 65, 1863-72) have become a standard for studies in dementia with Lewy Bodies (DLB), which show a very high specificity but low sensitivity. Clinical core features of DLB consist of rapid fluctuations in cognition, recurrent visual hallucinations and spontaneous and fluctuating features of Parkinsonism; these are further supported by high sensitivity for extrapyramidal side effects to neuroleptics and rapid eye movement sleep behaviour disorder. Dementia itself describes a syndrome characterised by memory impairment, intellectual deterioration, changes in personality and behavioural abnormalities.

Ubiquitin c-terminal hydrolase L1 (UCHL1) is one of the most abundant cytosolic proteins in the brain. In addition to neurons, it is expressed in testes (Wilkinson et al., 1989; Solano et al., 2000). Yet, UCHL1 is abnormally over-expressed in non-small-cell lung cancer (Hibi et al., 1999), pancreatic cancer (Tezel et al., 2000), colorectal cancer (Yamazaki et al., 2002) and myeloma cells (Otsuki et al., 2004). UCHL1 is an enzyme involved in the hydrolysis of polyubiquitin chains to increase the availability of free monomeric ubiquitin to the ubiquitin proteasome system (UPS), favouring protein degradation (Liu et al., 2002).

Several studies support the existence of a link between UCHL1 and certain degenerative disorders of the nervous system. An 193M mutation was described in UCHL1 gene in a German family with autosomal dominant Parkinson's disease (PD) (Leroy et al., 1998). This mutation leads to a 50% reduction of its hydrolytic activity in vitro (Lansbury and Brice, 2002). Moreover, UCHL1^{I93M} transgenic mice show loss of dopaminergic neurons in the substantia nigra and reduced dopamine content in the striatum at 20 weeks of age (Setsuie et al., 2007). Recent studies have shown classical Lewy pathology in a deceased sibling of a family affected by the 193M UCHL1 mutation who developed, in addition to DOPA-responsive Parkinsonism, marked cognitive deficits (Auburger et al., 2005). A possible link of UCHL1 with Alpha-synuclein pathology is supported by the observation that inhibition of UCHL1 activity in foetal rat ventral mesencephalic cultures is associated with Alpha-synuclein aggregates (McNaught et al., 2002). Increased intracellular aggregates containing ubiquitinated proteins have been found after UCHL1 inhibition by pro staglandins in human SK-N-SH cells (Li et al., 2004). These findings suggest that reduced UCHL1 activity impairs UPS function and protein degradation, thus facilitating, under appropriate conditions, the accumulation of abnormal protein aggregates. In line with this, reduced UCHL1 mRNA and protein is found in PD and in DLB, but only in brain regions in which aggregated proteins occur in the form of Lewy bodies and Lewy neurites (Barrachina et al., 2006).

There is a need for new methods and mechanisms for treating or preventing neurodegenerative diseases.

### BRIEF SUMMARY

In a first aspect the invention relates to a method of treating or preventing a neurodegenerative disease in a patient suffering from such a condition which comprises administering to such a patient a therapeutically effective amount of an agent that represses the transcriptional complex that represses the promoter of the UCHL1 gene.

In one embodiment, the agent affects the transcription, translation, subcellular localization or activity of one or several of the components of the transcriptional complex that represses the promoter of the UCHL1 gene.

In one embodiment, the neurodegenerative disease is a Lewy Body disorder.

The agent may be a HDAC inhibitor, such as Trichostatin A (TSA), Suberoylanilide hydroxamic acid (SAHA),

N-Hydroxy-4-(Methyl{[5-(2-Pyridinyl)-2-Thienyl]Sulfonyl}Amino)Benzamide,

4-Dimethylamino-N-(6-Hydroxycarbamoyethyl)Benzamide-N-Hydroxy-7-(4-Dimethylaminobenzoyl)Aminoheptanamide,

7-[4-(Dimethylamino)Phenyl]-N-Hydroxy-4,6-Dimethyl-7-Oxo-2,4-Heptadienamide, and Docosanol.

The agent may be a small molecule that inhibits the function of REST, sin3a, HDAC1, HDAC2, MeCP2, AOF2, RCOR1, JARID1C, BAF57, BAF170 and BRG1 in the transcriptional complex that represses the promoter of the UCHL1 gene. Alternatively the agent may be a small molecule that inhibits HDAC6.

In further embodiments the inhibition is provided by administering a small double stranded interference RNA (siRNA), a short hairpin RNA (shRNA), a microRNA, an antisense oligonucleotide or monoclonal antibodies directed against at least one of the genes that codes for one of the proteins belonging to the transcriptional repressor complex from UCHL1 gene, for example REST, sin3a, HDAC1, HDAC2, MeCP2, AOF2, RCOR1, JARID1C, BAF57, BAF170 and BRG1, or a different member of the aforementioned complex, or for a gene that codes for a protein that affects the transcription, translation, subcellular localization or activity of one or several of the components of the transcriptional complex that represses the promoter of the UCHL1 gene, for example HDAC6.

In one embodiment there is provided a method of screening for molecules that inhibit the transcriptional complex that represses the promoter of the UCHL1 gene comprising providing a cell line containing a reporter gene fused with the UCHL1 regulatory domains which expresses no or low levels of UCHL1, incubating the cell line with a molecule of interest and screening for expression of the reporter gene, wherein expression of the reporter gene indicates inhibition of the transcriptional complex that represses the promoter of the UCHL1 gene by the molecule of interest.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 shows the schematic representation of UCHL1 gene promoter. White boxes represent three putative DNA binding sites for NRSF/REST (NRSE) detected by MatInspector software. NRSE1 and NRSE3 are located in the complementary DNA chain (−) and the NRSE2 in the positive DNA chain (+). Exon 1 and 2 are indicated as dash boxes and intron 1 corresponds to grey box. The location of NRSE1, NRSE2 and NRSE3 sites is indicated relative to TATA signal located at position 268 of the sequence with GenBank number X17377. The transcription start site is shown with an arrow. Consensus: NT(T/C)AG(A/C)(A/G)CCNN(A/G)G(A/C)(G/S)AG (SEQ ID NO:1). NRSE1: 3′-GTGCGCAGCGCGGAGTGGTAC-5′ (SEQ ID NO:2). NRSE2: 5′-CACCGCTACCCGGAGAGCGCG-3′ (SEQ ID NO:3) NRSE3: 3′-ATCAGCGACTCGGCTCCCCCT-5′ (SEQ ID NO:4)

FIG. 2 shows NRSF/REST protein levels in frontal cortex homogenates in age-matched control cases (C, n=5), Parkinson's disease (PD, n=6), Dementia with Lewy Bodies, pure form (DLBp, n=6), and DLB common form (DLBc, n=7). A, NRSF/REST (121 KDa) and UCHL1 (25 KDa) protein levels are 30 detected by Western blot. S-Actin (45 kDa) is blotted to control protein loading. The image shows two samples from two different patients and age-matched controls but it is representative of all the samples indicated in the Table I. NRSF/REST protein levels are only seen in the frontal cortex of DLBp and DLBc. B, Densitometric analysis of NRSF/REST protein levels normalized with S-actin. AU: Arbitrary Units (mean±SD). *p<0.05 compared to control samples (ANOVA with post-hoc LSD test). NRSF/REST expression is compared with UCHL1 expression in the same cases to show an inverse relationship between NRSF/REST and UCHL1 in every case. The same results were observed in all cases summarized in Table I.

FIG. 3 shows NRSF/REST and UCHL1 expression levels in DMS53, U87-MG and HeLa cell lines. A, NRSF/REST and UCHL1 mRNA levels normalized with β-glucuronidase (GUSB). The detection was performed with TaqMan PCR as is indicated in experimental procedures section. B, NRSF/REST and UCHL1 protein levels detected by Western blotting. The figure shows the densitometric analysis of NRSF/REST and UCHL1 protein levels normalized with S-actin. AU: Arbitrary Units.

FIG. 4 shows the effect of NRSF/REST overexpression in DMS53 cell line. A, 1 μg of REEX1 vector, which encodes human full-length NRSF cDNA, was transfected in DMS53 cells using lipofectamine 2000. NRSF/REST mRNA and protein levels were increased after 48 h of REEX1 vector transfection. The protein levels were detected by Western Blot showing two independent transfections. S-Actin (45 kDa) is blotted to control protein loading. The over-expression of NRSF/REST transcription factor reduces endogenous UCHL1 (B) and synaptophysin (C) mRNA levels. REEX1 over-expression was performed in triplicate (6-well plates) in three independent experiments. The mRNA levels of all the analysed genes were detected by TaqMan PCR and the endogenous control used was β-glucuronidase.

FIG. 5 shows the effect of NRSF/REST siRNA transfection in U87-MG cell line. A, NRSF/REST protein levels are detected by Western Blot after 48h of NRSF/REST siRNA transfection (siRNA#1 and siRNA#2). The scramble siRNA transfection does not modify the expression of endogenous NRSF/REST levels. S-Actin (45 kDa) is blotted to control protein loading. Reduction of NRSF/REST transcription factor increases endogenous UCHL1 (B) and synaptophysin (C) mRNA levels. siRNA transfection was carried out in triplicate (6-well plates) in three independent experiments. The mRNA levels of all the analysed genes were detected by TaqMan PCR and the endogenous control used was f3-glucuronidase (GUSB). AU: Arbitrary Units (mean±SD). *p<0.01 and **p<0.001 compared with non-transfected cells (ANOVA with post-hoc LSD test).

FIG. 6 shows a chromatin immunoprecipitation (ChIP) assay using NRSF/REST antibody in HeLa, U87-MG and DMS53 cell lines illustrating that NRSF/REST binds the UCHL1 regulatory region. A, Schematic representation of minimal UCHL1 gene promoter. Grey boxes represent the three putative DNA binding sites for NRSF/REST (NRSE). The transcription start site is indicated as +1. Immunoprecipitated DNA was analysed by PCR using two sets of primers that amplified a 247 by region of the UCHL1 gene promoter which encompasses an NRSE site (NRSE1) and a 214 by region which contains the other two NRSE sites (NRSE2 and NRSE3). B, ChIP assay was performed in U87-MG cells using a goat antibody anti-human NRSF/REST and a rabbit polyclonal anti-human acetyl-histone 3 (Lys9) antibody as positive control. The ChIP assay with goat serum was performed as a negative control. Primers set number 1 amplify a 247 by region of the UCHL1 gene promoter and primers set number 2 amplify a 214 by region as schematically indicated above. The same results were obtained using HeLa cells. C, The same ChIP analysis performed but using DMS53 cells. Input refers to DNA chromatin not immunoprecipitated with the specific antibody. ChIP refers to DNA chromatin immunoprecipitated with the specific antibody. M: 100 by DNA ladder marker.

FIG. 7 shows the effect of the application of siRNAs and miRNAs directed against the repressor complex on the levels of UCHL1 mRNA expression in U87-MG cells. Treatment with 100 nM of total siRNA or miRNA concentration during 48 h led to over 2 fold induction of expression was when siRNAs directed against MeCP2, RCOR1, sin3A, HDAC1, HDAC2, and AOF2 were employed (direct inhibition of components the repression complex). In this experiment, treatment with 100 nM of siRNA against HDAC6 (indirect inhibition of components the repression complex) led to an induction of nearly 7 fold in UCHL1 mRNA expression. Lower inductions (25%) were observed with siRNAs directed against REST and JARID1C and the pool of miRNAs

FIG. 8 shows the effect of the application of 24h treatment with 100 mM of the HDAC inhibitor trichostatin A (TSA) on the expression of UCHL1 in U87-MG (A) and HeLa (B) cells.

FIG. 9 shows that no systematic difference in DNA methylation of the minimal UCHL1 gene promoter in post-mortem cortical brain samples of patients with Dementia with Lewy Bodies (pure and common form) and age-matched controls can be detected. A, Partial consensus sequence of UCHL1 gene promoter (GenBank accession number X17377) with nucleotides numbered relative to ATG (+1) (Consensus sequence −248 CAGATTATCTCACCGGCGAGTGAGACTGCAAGGTTTGGGGGCCCGGCC GTACCACTCCGCGCTGCGCACGGGGGGTTCGTACCC −174) (SEQ ID NO:5). Nine CpG islands are indicated with boxes (the completed promoter sequence contains 35 CpG sites). B, Chromatogram of a partial PCR product from bisulfite treated UCHL1 gene promoter (−248 to −174) of a 79-year-old male (−248 TAGATTATTTTATCGGTGAGTGAGATTGTAAGGTTTGGGGGTTTGGTTGTAT TATTTTGTGTTGCGTATGGGGGGTTTGTATTT −174) (SEQ ID NO:6). C, Summary of all clones determined by bisulfite sequencing analysis of UCHL1 gene promoter from age-matched controls (n=5), dementia with Lewy bodies pure form (DLBp, n=6) and common form (DLBc, n=7). Every circle corresponds to the 35 CpG sites described in UCHL1 gene promoter (Bittencourt-Rosas et al., 2001). Non-methylated cytosines (white circles) and methylated cytosines (black circles) are indicated. The number of clones with methylated cytosines is indicated with respect to the total number of clones analysed for each sample. Sample number corresponds to that found in Table 1.

FIG. 10 shows the inductory effect of the treatment of HeLa and U87-MG cells with the demethylation agent 5-azacytidine on UCHL1 expression.

### DETAILED DESCRIPTION

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

The biological role of UCHL1 and its role in pathogenesis encouraged the present study focused on the transcriptional regulation of UCHL1. One report has demonstrated that the UCHL1 gene promoter contains 35 CpG islands which are fully methylated in UCHL1 non-expressing HeLa cells (Bittencourt-Rosas et al., 2001). For this reason, we have analysed the methylation status of the UCHL1 gene promoter in human post-mortem frontal cortex samples of patients with DLB. Because of the negative results, we then carried out a bioinformatic search employing the Genomatix MatInspector software to identify putative transcription factors binding sites in the UCHL1 gene promoter. Among the promoter elements, we identified 1 weak binding site for REST neuron-restrictive silencer factor/RE-1 silencer transcription factor (NRSF/REST) in the promoter and 2 weak binding sites in the first intron. NRSF/REST has been defined as a neuronal gene repressor in non-neuronal cells (Chong et al., 1995; Schoenherr and Anderson, 1995).

However, the potential NRSF/REST binding sites detected in the UCHL1 gene promoter and first intron had low matrix similarity scores (0.69; 0.71 and 0.67 respectively) and the “Explanations of Scores” for the Matlnspector software define a “good” match as one having a matrix similarity of >0.80 (Table I). Therefore it was not predicted from this data that REST would bind to the UCHL1 gene promoter.

Table 1. Summary of the main clinical and neuropathological findings in the present series.

Parkinson's disease (PD), Diffuse Lewy body disease: Dementia with Lewy bodies, pure form (DLBp) and common form (DLBc), and controls. M: male, F: female. NFT: neurofibrillary tangle. P-m delay: post-mortem delay in hours. Braak stages refer to Braak and Braak Alzheimer's disease (AD) changes (Braak and Braak, 1999). Staging of H-synuclein pathology (Lewy bodies and Lewy neurites) related to sporadic Parkinson's disease (PD) was done according to Braak et al., 2003.

| | | | | Post | Braak | SA4 | Braak |
|---|---|---|---|---|---|---|---|
| | | | | Mortem | stages | amyloid | stages |
| Case | Disease | Gender | Age | delay | AD | NFT | PD |
| 1 | Control | F | 80 | 3 | 0 | 0 | 0 |
| 2 | Control | F | 73 | 5 | 0 | 0 | 0 |
| 3 | Control | M | 79 | 7 | 0 | II | 0 |
| 4 | Control | F | 75 | 6 | 0 | 0 | 0 |
| 5 | Control | F | 82 | 11 | A | III | 0 |
| 6 | PD | M | 66 | 5 | 0 | I | 3 |
| 7 | PD | M | 81 | 5 | A | II | 4 |
| 8 | PD | M | 88 | 2 | 0 | II | 4 |
| 9 | PD | M | 57 | 11 | 0 | 0 | 4 |
| 10 | PD | F | 60 | 4 | A | 0 | 4 |
| 11 | PD | F | 70 | 4 | 0 | 0 | 4 |
| 12 | DLBp | M | 60 | 8 | A | I | 6 |
| 13 | DLBp | F | 70 | 8 | 0 | 0 | 5 |
| 14 | DLBp | M | 71 | 6 | B | 0 | 5 |
| 15 | DLBp | F | 72 | 3 | A | 0 | 5 |
| 16 | DLBp | M | 85 | 7 | B | II | 6 |
| 17 | DLBp | F | 77 | 5 | B | 0 | 5 |
| 18 | DLBc | M | 78 | 6 | C | V | 6 |
| 19 | DLBc | M | 78 | 7 | C | V | 6 |
| 20 | DLBc | M | 71 | 5 | C | V | 5 |
| 21 | DLBc | M | 77 | 5 | C | VI | 6 |
| 22 | DLBc | M | 84 | 4 | C | VI | 5 |
| 23 | DLBc | M | 78 | 13 | C | IV | 5 |
| 24 | DLBc | F | 91 | 5 | C | V | 6 |

In addition UCHL1 was not identified previously as a target for REST/UCHL1 in genome wide analysis, for example:

UCHL1 is not included as a target for the RE1 target database (http://www.bioinformatics.leeds.ac.uk/REldb_mkII/) at any PSSM cutoff level (Genome-wide analysis of repressor element 1 silencing transcription factor/neuron-restrictive silencing factor (REST/NRSF) target genes, Bruce et al, Proc Natl Acad Sci USA. 2004 Jul. 13; 101(28): 10458-10463).

UCHL1 is not included as a target in the supplementary tables in Otto S J et al. A new binding motif for the transcriptional repressor REST uncovers large gene networks devoted to neuronal functions. J Neurosci. 2007 Jun. 20; 27(25):6729-39.

UCHL1 is not included in the supplementary tables of Johnson D S et al. Genome-wide mapping of in vivo protein-DNA interactions. Science. 2007 Jun. 8; 316(5830):1497-502. Epub 2007 May 31.

We evaluated REST expression in PD and DLB and found that REST was slightly overexpressed. Its expression is inversely related to the UCHL1 expression levels in the frontal cortex of pure and common DLB cases. In addition; we reviewed data published on incipient AD (Blalock E M et al. Incipient Alzheimer's disease: microarray correlation analyses reveal major transcriptional and tumor suppressor responses. Proc Natl Acad Sci USA. 17 Feb. 2004; 101(7):2173-8. Epub 9 Feb. 2004). Although not specifically cited in Table 4, dedicated to transcription factors associated with incipient AD; we found a 35% increase was for the expression level of REST and a 35% decrease in the UCHL1 level of severe AD cases in Supporting Table 5 of the cited article; indicating that the relationship between NRSF/REST may also exist in AD.

| | Control | Incipient | Moderate | Severe |
|---|---|---|---|---|
| REST | 145 ± 11 | 141 ± 13 | 162 ± 21 | 194 ± 14 |
| UCHL1 | 6962 ± 994 | 5602 ± 1002 | 3932 ± 611 | 4352 ± 1364 |

After performing several functional analyses with three cell lines, we confirmed that NRSF/REST binds to the promoter of UCHL1 and regulates the expression of UCHL1. Together, these findings demonstrate NRSF/REST as a relevant transcription factor that negatively regulates UCHL1 expression and causes downregulation of expression in diseases with Lewy bodies, including PD and DLB.

As a result of this new understanding of the role of NRSF/REST in the down regulation of UCHL1 gene expression and the known importance of UCHL gene expression in DLB, other Lewy body disorders and other neurodegenerative diseases, we are able to propose several new and inventive approaches to the treatment and prevention of neurodegenerative diseases.

According to the invention there is provided a method of treating or preventing a neurodegenerative disease in a patient suffering from such a condition which comprises administering to such a patient a therapeutically effective amount of an agent that represses the transcriptional complex that represses the promoter of the UCHL1 gene.

Specific neurodegenerative diseases include Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington's disease, HIV-associated dementia, Kennedy's disease, Krabbe disease, dementia with Lewy bodies, Machado-Joseph disease (Spinocerebellar ataxia type 3), Multiple sclerosis, Multiple System Atrophy, Neuroborreliosis, Parkinson's disease, Parkinson's disease with dementia, Pelizaeus-Merzbacher Disease, Pick's disease, Primary lateral sclerosis, Prion diseases, Refsum's disease, Sandhoff disease, Schilder's disease, Schizophrenia, Spinocerebellar ataxia (multiple types), Spinal muscular atrophy, Steele-Richardson-Olszewski disease, and Tabes dorsalis. The method of the invention is particularly suitable for treating Alzheimer's disease, Huntington's disease and Lewy Body disorders, such as dementia with Lewy bodies, Parkinson's disease, Parkinson's disease with dementia.

By “treating or preventing” we mean symptomatic improvement, which may include enhanced cognition, more autonomy and/or improvement in neuropsychiatric and behavioural dysfunction; and/or disease modification with slowing or arrest of symptom progression of the dementing process; and/or primary prevention of disease by intervention in key pathogenic mechanisms at a pre-symptomatic stage.

A Lewy body disorder is defined herein as a condition which is characterised by disorders of alpha-synuclein metabolism, which gives rise to the formation of abnormal neuronal alpha-synuclein inclusions. These are the defining pathologic process common to both PDD and DLB. More particularly Lewy body disorders include dementia with Lewy bodies (DLB), Parkinson's disease (PD) and PD with dementia (PDD).

The methods of the invention are particularly suitable for treating a neurodegenerative disease which is characterised by the overexpression of REST. In other words, REST can be used as a biomarker for the detection of a neurodegenerative disease which could be treated by the method of the invention. AD is an example of such a condition, where REST is slightly upregulated in the cortex and UCHL1 is slightly downregulated.

In this respect we have identified two models which may benefit from the treatments proposed herein. The first model develops Lewy neurites (mouse overexpressing a synuclein with the A53T mutation) and could be envisaged as a model for Lewy Body disease, whilst the second model benefits from recombinant UCHL1 protein therapy. In the second model (APP/PS 1 mouse model), the delivery of an exogenous TAT-UCHL1 fusion protein improves the symptoms of the neurodegeneration, supporting the role for UCHL1 in therapeutic strategies in neurodegenerative disease.

Further, a number of animal model systems for Huntington's disease are available. See, e.g., Brouillet (2000) Functional Neurology 15(4):239-251; Ona et al. (1999) Nature 399:263-267; Bates et al. (1997) Hum. Mol. Genet. 6(10):1633-1637; Hansson et al. (2001) J. Neurochem. 78:694-703; and Rubinsztein (2002) Trends Genet. 18:202-209 (a review on various animal and non-human models of HD).

One transgenic mouse model for Huntington's disease is the R6/2 model (Mangiarini et al. (1996) Cell 87:493-506). The R6/2 mice over-express exon 1 of the human Huntingtin gene which has an expanded CAG/polyglutamine repeat lengths (150 CAG repeats on average). These mice develop a progressive, ultimately fatal, neurological disease with many biochemical and physiological features of human Huntington's disease. For example, abnormal aggregates, constituted in part by the N-terminal part of Huntingtin (encoded by HD exon 1), are observed in R6/2 mice, both in the cytoplasm and nuclei of cells (Davies et al. (1997) Cell 90:537-548). These transgenic mice are characterized by reduced weight gain, reduced lifespan, and motor impairment characterized by abnormal gait, resting tremor, hindlimb clasping, and hyperactivity from 8 to 10 weeks after birth (for example the R6/2 strain; see Mangiarini et al. (1996) Cell 87:493-506). The phenotype worsens progressively toward hypokinesia. The brains of these transgenic mice demonstrate neurochemical and histological abnormalities, such as changes in neurotransmitter receptors (glutamate, dopaminergic), decreased concentration of N-acetylaspartate (a marker of neuronal integrity), and reduced striatum and brain size. Thus, the compounds of the invention can be evaluated in this model by assessing parameters related to neurotransmitter levels, neurotransmitter receptor levels, brain size, striatum size, life-span, biochemical disease evidence (e.g., abnormal aggregates), and motor impairment.

In one embodiment, the methods of treatment of the invention will be applied to patients that have downregulated expression of the UCHL1 protein. In the disease conditions specified above, it may be preferable to verify an intact UCHL1 CDS prior to treatment, since overexpression of deficient UCHL1 enzymes, like the 193M allele may be contraindicated. In other words, patient stratification may be advantageous.

In one embodiment the transcriptional complex which is repressed is the NRSF/REST complex. This complex comprises REST, sin3a, HDAC1, HDAC2, MeCP2, AOF2, RCOR1, JARID1C, SMARCE1 (BAF57), SMARCA4 (BRG1) and SMARCC2 (BAF170) and other components. The transcriptional complex may be repressed directly or indirectly by altering the transcription, translation, subcellular localisation or activity of one or several components of the complex. For example, inhibition of HDAC6 which is involved in the retention of the components of the complex in the cytoplasm can successfully inhibit the transcriptional complex.

This complex may be inhibited by various methods. For example in one embodiment the REST complex may be inhibited by HDAC inhibitors. Histone deacetylase (HDAC) plays a role in transcriptional regulation and catalyses the deacetylation of lysine residues located in the NH(2) terminal tails of histones and non-histone proteins. They play an important role in the regulation of the expression status of genes. Further HDACs are found in the REST transcriptional complex.

Histone deacetylases (HDACs) are divided into three classes: class I HDACs (HDACs 1, 2, 3, and 8), similar to yeast RPD3 and localized in the nucleus; class II HDACs (HDACs 4, 5, 6, 7, 9, and 10); homologous to yeast HDA1 protein and localized both the nucleus and cytoplasm; and class III HDACs, a structurally distinct class of NAD-dependent enzymes similar yeast SIR2.

HDAC inhibitors are small molecules that target histone deacetylases. The application of HDAC inhibitors can reverse the silencing of genes generated by the acetylation of histones; and has been proposed for reactivating silenced tumours suppressor genes in cancer.

Accordingly, with this new understanding of the regulation of the UCHL1 promoter and the relevance of UCHL1 expression in the development of the disease, HDAC inhibitors would be expected to derepress the UCHL1 gene and increase the expression of UCHL1. As such, they would be useful in the treatment or prevention of neurodegenerative diseases, and particularly in the treatment of Lewy body disorders.

HDAC inhibitors that can be used in this first aspect of the invention include inhibitors against any HDAC, including for example inhibitors against HDAC1, HDAC2 or HDAC6. Examples of such inhibitors include: Trichostatin A (TSA),Suberoylanilide hydroxamic acid (SAHA),N-Hydroxy-4-(Methyl{[5-(2-Pyridinyl)-2-Thienyl]Sulfonyl}Amino)Benzamide,4-Dimethylamino-N-(6-Hydroxycarbamoyethyl)Benzamide-N-Hydroxy-7-(4-Dimethylaminobenzoyl)Aminoheptanamide,7- [4-(Dimethylamino)Phenyl]-N-Hydroxy-4,6-Dimethyl-7-Oxo-2,4-Heptadienamide, Docosanol, (S)-[5-Acetylamino-1-(2-oxo-4-trifluoromethyl-2H-chromen-7-ylcarbamoyl) pentyl]carbamic acid tert-butyl ester (BATCP), Benzyl ((S)-[1-(4-methyl-2-oxo-2H-chromen-7-ylcarbamoyl)-5-propionyl aminopentyl]carbamate (MOCPAC), and 4-(Dimethylamino)-N-[7-(hydroxyamino)-7-oxoheptyl]-benzamide (M344).

Preferred HDAC inhibitors include the carboxylic acid class of HDAC inhibitors and derivatives thereof. In one aspect, the HDAC inhibitor is a short-chain to medium-chain fatty acid or a derivative or analog thereof. Examples of short chain fatty acids include, but are not limited to, butyric acid, phenylalkanoic acids, phenylbutyrate (PB), 4-phenylbutyrate (4-PBA), pivaloyloxymethyl butyrate (Pivanex, AN-9), isovalerate, valerate, valproate, valproic acid, propionate, butyramide, isobutyramide, phenylacetate, 3-bromopropionate, or tributyrin. Short-chain fatty acid compounds are described e.g., in U.S. Pat. Nos. 4,988,731; 5,212,326; 4,913,906; 6,124,495; 6,110,970; 6,419,953; 6,110,955; 6,043,389; 5,939455; 6,511,678; 6,528,090; 6,528,091; 6,713,086; 6,720,004; U.S. Patent Publication No. 20040087652, Intl. Publication No. WO 02/007722, and in Phiel et al., J Biol Chem., 276(39):36734-41 (2001), Rephaeli et al., Int J Cancer., 116(2):226-35 (2005), Reid et al., Lung Cancer., 45(3):381-6 (2004), Gottlicher et al., 2001, EMBO J., 22(13):341 1-20 (2003), and Vaisburg et al., Bioorg Med Chem Lett., 14(1):283-7 (2004). Other short to medium chain carboxylic acids include, but are not limited to, those disclosed in e.g., U.S. Pat. No. 7,176,240; WO 98/22436; and WO 2004/110974.

Preferred inhibitors are orally administrable and capable of passing the blood brain barrier such as SAHA; and should at least release the repression of the UCHL1 promoter; which would be the control for effectiveness. In some embodiments, the preferred inhibitors have at least 10%, 20%, or 30% or more blood brain barrier penetration.

In another embodiment the REST complex may be inhibited by agents that inhibit the function of the other members of the repression complex, including REST, MeCP2, mSin3a, AOF2, RCOR1, JARID1C, BAF57, BAF170 and BRG1. Such agents may act by preventing the transcriptional repression complex from binding to the gene promoter or may act by preventing members of the complex from interacting with each other. In either case the end result will be that the complex is prevented from inhibiting gene expression, so the gene, UCHL1 will become derepressed.

Examples of suitable small molecules include: Benzyl ((S)-[1-(4-methy1-2-oxo-2H-chromen-7-ylcarbamoyl)-5-propionylaminopentyl]carbamate; Molecular Formula: C27H31N3O6; (S)-[5-Acetyl amino-1-(2-oxo-4-trifluoromethyl-2H-chromen-7-ylcarbamoyl)pentyl]carbamic acid tert-butyl ester; Molecular Formula: C23H28F3N3O6; 4-(Dimethylamino)-N-[7-(hydroxyamino)-7-oxoheptyl]-benzamide; N-Hydroxy-7-(4-dimethylaminobenzoyl)-aminoheptanamide. (all available from Sigma)

In one embodiment of the invention, the histone deacetylase inhibitor has a structure of (R)-(A)-(L)-C═(O)NHOH wherein (A) is a carbocyclic, aryl, or heterocyclic ring system substituted with one or more R groups, and L is a linker group. In compounds having this general formula it is believed that the hydroxamate group functions as a metal binding group that interacts with the metal ion at the active site of the HDAC enzyme. The A ring system is believed to be at the entrance to the metal ion pocket in the active site. Non-limiting examples of heterocyclic, carbocyclic, and aryl ring systems, along with various linkers are given in the specific exemplified compounds below.

In one preferred embodiment the HDAC inhibitor is chosen from N-Hydroxy 2-(5-naphthalen-2-ylmethylhexahydropyrrolo[3,4-c]pyrrol-2 [1H]-yl)pyrimidine-5-carboxamide; N-Hydroxy 2-{6-[(2-naphthylsulfonyl)amino]-3-azabicyclo[3.1.0]hex-3-yl}pyrimidine-5-carboxamide trifluoroacetate; N-Hydroxy 2-{6-[(6-fluoroquinolin-2-ylmethyl)amino]-3-aza-bicyclo[3.1.0]hex-3-yl}pyrimidine-5-carboxamide; N-Hydroxy 2-[5-(naphthalene-2-carbonyl)-hexahydropyrrolo[3,4-c]pyrrol-2-yl]pyrimidine-5-carboxamide; (S)-[4-(7-Hydroxycarbamoyl-heptanoylamino)-benzylamino]-phenyl-acetic acid cyclopentyl ester; (S)-2-[3-(7-Hydroxycarbamoyl-heptanoylamino)-benzylamino]-4-phenyl-butyric acid cyclopentyl ester; (S)-[3-(7-Hydroxycarbamoyl-heptanoylamino)-benzylamino]-phenyl-acetic acid cyclopentyl ester; (4-{[(2-Hydroxycarbamoyl-benzo[b]thiophen-6-ylmethyl)-amino]-methyl}-benzylamino)-acetic acid cyclopentyl ester, (S)-2-Amino-4-(4-{[(2-hydroxycarbamoyl-benzo[b]thiophen-6-ylmethyl)-amino]-methyl}-phenoxy)-butyric acid cyclopentyl ester; (S)-2-Amino-5-(4-{[(2-hydroxycarbamoyl-benzo[b]thiophen-6-ylmethyl)-amino]-methyl}-phenoxy)-pentanoic acid cyclopentyl ester; (R)-2-Amino-4-(4-{[(2-hydroxycarbamoyl-benzo[b]thiophen-6-ylmethyl)-amino]-methyl}-phenoxy)-butyric acid cyclopentyl ester; 2-(S)-Amino-3-[4-(4-{[(2-hydroxycarbamoyl-benzo[b]thiophen-6-ylmethyl)-amino]-methyl}-phenoxy)-cyclohexyl]-propionic acid cyclopentyl ester, and pharmaceutically acceptable salts thereof.

In a further preferred embodiment of the invention the HDAC inhibitor is a carboline or beta-carboline derivative wherein the carboline or betacarboline ring systems (or analogs thereof) have a hydroxamate or N-hydroxy acylamino metal binding group as a pendant group, and pharmaceutically acceptable salts thereof.

In a yet further preferred embodiment of the invention, the HDAC inhibitor is a benzoimidazole derivative. In one aspect of this embodiment, the benzoimidazole derivative is chosen from 3-[1-(3-Dimethylamino-2,2-dimethyl-propyl)-2-(2,2-dimethyl-propyl)-1H-benzoimidazol-5-yl]-N-hydroxy-acrylamide; 3-[1-(3-Dimethylamino-2,2-dimethyl-propyl)-2-isopropyl-1H-benzoimidazol-5-yl]-N-hydroxy-acrylamide; 3-[2-Butyl-1-(3-dimethylamino-2,2-dimethyl-propyl)-1H-benzoimidazol-5-yl]-N-hydroxy-acrylamide; 3-[1-(3-Dimethylamino-2,2-dimethyl-propyl)-2-(2-methylsulfanyl-ethyl)-1H-benzoimidazol-5-yl]-N-hydroxyl-acrylamide; 3-[2-(2,2-Dimethyl-propyl)-1-(2-isopropylamino-ethyl)-1-H-benzoimidazol-5-yl]-N-hydroxy-acrylamide; 3-[1-(2-Diisopropylamino-ethyl)-2-(2,2-dimethyl-propyl)-1-H-benzoimidazol-5-yl]-N-hydroxy-acrylamide; 3-[1-(2-Diisopropylamino-ethyl)-2-isobutyl-1H-benzoimidazol-5-yl]-N-hydroxy-acrylamide; 3-[1-(3-Dimethylamino-2,2-dimethyl-propyl)-2-hex-3-enyl-1-H-benzoimidazol-5-yl]-N-hydroxy-acrylamide; 3-[1-(3-Dimethylamino-2,2-dimethyl-propyl)-2-(2,4,4-trimethyl-pentyl)-1H-benzoimidazol-5-yl]-N-hydroxy-acrylamide; 3-[2-Cyclohexyl-1-(3-dimethylamino-2,2-dimethyl-propyl)-1H-benzoimidazol-5-yl]-N-hydroxyacrylamide; 3-[2-Bicyclo[2.2.1]hept-5-en-2-yl-1-(3-dimethylamino-2,2-dimethyl-propyl)-1H-benzoimidazol-5-yl]-N-hydroxy-acrylamide; 3-[1-(2-Diethylamino-ethyl)-2-hex-3-enyl-1H-benzoimidazol-5-yl]-N-hydroxy-acrylamide; 3-[1-(2-Diisopropylamino-ethyl)-2-hex-3-enyl-1H-benzoimidazol-5-yl]-N-hydroxy-acrylamide; 3-[2-Hex-3-enyl-1-(2-isopropylamino-ethyl)-1H-benzoimidazol-5-yl]-N-hydroxy-acrylamide; 3-[2-Hex-3-enyl-1-(3-isopropylamino-propyl)-1H-benzoimidazol-5-yl]-N-hydroxy-acrylamide; 3-[1-(2-Ethylamino-ethyl)-2-hex-3-enyl-1H-benzoimidazol-5-yl]-N-hydroxya-crylamide; 3-[1-(2-Diethylamino-ethyl)-2-hexyl-1H-benzoimidazol-5-yl]-N-hydroxy-acrylamide; 3-[2-[3,3-Dimethyl-butyl)-1-(2-Dimethylamino-ethyl)-1H-benzoimidazol-5-yl]-N-hydroxy-acrylamide; 3-[1-(2-Dimethylamino-ethyl)-2-pentyl-1H-benzoimidazol-5-yl]-N-hydroxy-acrylamide; 3-[1-(2-Dimethylamino-ethyl)-2-(2,2,2-trifluoro-ethyl)-1H-benzoimidazol-5-yl]-N-hydroxy-acrylamide; N-Hydroxy-3-[1-(5-methyl-1-H-pyrazol-3-yl)-2-(2,4,4-trimethyl-pentyl)-1H-benzoimidazol-5-yl]-acrylamide; 3-[1-(2-Ethylamino-ethyl)-2-pentyl-1H-benzoimidazol-5-yl]-N-hydroxy-acrylamide 3-(2-Butyl-1-pyrrolidin-3-yl-1H-benzoimidazol-5-yl)-N-hydroxy-acrylamide; 3-(2-Butyl-1-piperidin-4-yl-1H-benzoimidazol-5-yl)-N-hydroxy-acrylamide; 3-[2-(4-Cyano-butyl)-1-(2-diethylamino-ethyl)-1H-benzoimidazol-5-yl] -N-hydroxy-acrylamide; (E)-3-(1-(1-butylpiperidin-3-yl)-1H-benzo[d]imidazol-5-yl)-N-hydroxy-acrylamide; (E)-N-hydroxy-3-(1-(1-(pent-4-enyl)piperidin-3-yl)-1H-benzo [d] imidazol-5-yl)acrylamide; (E)-3-(1-(1-(3-dimethylbutyl)piperidin-4-yl)-1H-ben2o[d]imidazol-5-yl)-N-hydroxy-acrylamide; 3-[1-(2-Diethylamino-ethyl)-2-propylamino-1H-benzoimidazol-5-yl]-N-hydroxy-acrylamide; (E)-N-hydroxy-3-(1-(2-(isopropyl(propyl)amino)ethyl)-1H-benzo[d]imidazol-5-yl)acrylamide; 3-{1-[2-(Butyl-isopropyl-amino)-ethyl]-1H-benzoimidazol-5-yl}-N-hydroxy-acrylamide; 3-(1-{2-[(2-Ethyl-butyl)-methyl-amino]-ethyl}-1H-benzoimidazol-5-yl)-N-hydroxy-acrylamide; (E)-3-(1-(2-(bis(3,3-dimethylbutyl)amino)ethyl)-1H-benzo[d]imidazol-5-yl)-N-hydroxy-acrylamide; (E)-3-(1-(2-(diisobutylamino)ethyl)-1H-benzo[d]imidazol-5-yl)-N-hydroxy-acrylamide; 3-{1-[2-(3,3-Dimethyl-butylamino)-ethyl]-1H-benzoimidazol-5-yl}-N-hydroxy-acrylamide; N-Hydroxy-3-{1-[2-(methyl-pent-4-enyl-amino)-ethyl]-1H-benzoimidazol-5-yl}-acrylamide; 3-(1-{2-[(2,2-Dimethyl-propyl)-propyl-amino]-ethyl}-1H-benzoimidazol-5-yl)-N-hydroxy-acrylamide; 3-{1-[2-(3,3-Dimethyl-butylamino)-ethyl]-2-ethyl-1H-benzoimidazol-5-yl}-N-hydroxy-acrylamide; 3-(1-{2-[(3,3-Dimethyl-butyl)-methyl-amino]-ethyl}-2-propyl-1H-benzoimidazol-5-yl)-N-hydroxy-acrylamide; 3-(1-{2-[(3,3-Dimethyl-butyl)-(2,2,2-trifluoro-ethyl)-amino]-ethyl}-1H-benzoimidazol-5-yl)-N-hydroxy-acrylamide; 3-(1-{2-[Butyl-(2,2,2-trifluoro-ethyl)-amino]-ethyl}-1H-benzoimidazol-5-yl)-N-hydroxy-acrylamide; and pharmaceutically acceptable salts thereof.

In a yet further preferred aspect of the invention the HDAC inhibitor is an imidazo[1,2-A]pyridine derivative. In one aspect of this embodiment, the imidazo[1,2-A]pyridine derivative is chosen from (E)-N-hydroxy-3-(2-phenethyl-3-(3,4,5-trimethoxyphenylamino)imidazo[1,2-a]pyridin-6-yl)acrylamide; (E)-N-hydroxy-3-(2-phenethyl-3-(3,4,5-trimethoxyphenylamino)imidazo[1,2-a]pyridin-6-yl)acrylamide; (E)-3-(3-(benzo[d][1,3]dioxol-5-ylmethylamino)-2-phenethylimidazo[1,2-a]pyridin-i3-yl)-N-hydroxyacrylamide; N˜Hydroxy-3-[2-phenethyl-3-(4-piperidin-1-yl-phenylamino)-imidazo[1,2-a]pyridin-6-yl]-acrylamide; 3-(2-Hexyl-3-{2-[(2-hydroxy-ethyl)-propyl-carbamoyl]-ethylamino}-imidazo[1,2-a]pyridin-7-yl)-N-hydroxy-acrylamide; N-Hydroxy-3-(2-phenyl-imidazo[1,2-a]pyridin-7-yl)-acrylamide; 3-(3-Butylaminomethyl-2-phenyl-imidazo[1,2-a]pyridin-7-yl)-N-hydroxy-acrylamide; N-Hydroxy-3-{3-[(methyl-propyl-amino)-methyl]-2-phenyl-imidazo[1,2-a]pyridin-7-yl}-acrylamide; N-Hydroxy-3-(2-methyl-imidazo[1,2-a]pyridin-7-yl)-acrylamide; 3-(3-Butylaminomethyl-2-methyl-imidazo [1,2-a]pyridin-7-yl)-N-hydroxy-acrylamide; 3-{2-tert-Butyl-3-[(2-diethylamino-ethylamino)-methyl]-imidazo[1,2-a]pyridin-7-yl}-N-hydroxy-acrylamide; 3-(3-{[(2-Dimethylamino-ethyl)-ethyl-amino]-methyl}-2-phenyl-imidazo[1,2-a]pyridin-7-yl)-N-hydroxy-acrylamide; N-Hydroxy-3-(2-phenyl-3-pyrrolidin-1-ylmethyl-imidazo[1,2-a]pyridin-7-yl)-acrylamide; 3-{3-[(Cyclopropylmethyl-amino)-methyl]-2-phenyl-imidazo[1,2-a]pyridin-7-yl}-N-hydroxy-acrylamide; 3-(3-Cyclopropylaminomethyl-2-phenyl-imidazo[1,2-a]pyridin-7-yl)-N-hydroxy-acrylamide; 3-[3-Butylaminomethyl-2-(4-fluoro-phenyl)-imidazo[1,2-a]pyridin-7-yl]-N-hydroxy-acrylamide; 3-[3-(tert-Butylamino-methyl)-2-(4-fluoro-phenyl)-imidazo[1,2-a]pyridin-7-yl]-N-hydroxy-acrylamide, and pharmaceutically acceptable salts thereof.

In a yet further preferred embodiment of the invention, the HDAC inhibitor is a benzothiothene derivative. In one aspect of the invention the benzothiothene derivative is chosen from 5-Phenylacetylamino-benzo[b]thiophene-2-carboxylic acid hydroxyamide; 5-Benzoylamino-benzo[b]thiophene-2-carboxylic acid hydroxyamide; 5-(3-Phenyl-acryloylamino)-benzo[b]thiophene-2-carboxylic acid hydroxyamide; 5-[3-Phenyl-2-(toluene-4-sulfonylamino)-propionylamino]-benzo[b]thiophene-2-carboxylic acid hydroxyamide; 5-[2-(3,4-Dimethoxy-phenyl)-acetylamino]-benzo[b]thiophene-2-carboxylic acid hydroxyamide; Tetrahydro-furan-2-carboxylic acid [1-(2-hydroxycarbamoyl-benzo[b]thiophen-5-ylcarbamoyl)-2-phenyl-ethyl]-amide; Tetrahydro-furan-3-carboxylic acid [1-(2-hydroxycarbamoyl-benzo[b]thiophen-5-ylcarbamoyl)-2-phenyl-ethyl]-amide; 5-{2-[3-(2,5-Dimethoxy-benzyl)-ureido]-3-phenyl-propionylamino}-benzo[b]thiophene-2-carboxylic acid hydroxyamide; 5-[3-Phenyl-2-(3-thiophen-2-ylmethyl-ureido)-propionylamino]-benzo[b]thiophene-2-carboxylic acid hydroxyamide; 5-[(3,3-Dimethyl-butyryl)-(2-isopropylamino-ethyl)-amino]-benzo[b]thiophene-2-carboxylic acid hydroxyamide, and pharmaceutically acceptable salts thereof.

In a yet further preferred embodiment of the invention, the HDAC inhibitor is a depsipeptide or a derivative or analog of thereof. In one aspect of this embodiment, the depsipeptide is chosen from is FK228 and Spiruchostatin A. In one aspect of this embodiment, the depsipeptide analog or derivative is an amino acid derivative or an analog of FK228 or Spiruchostatin A, and pharmaceutically acceptable salts thereof.

In a yet further preferred embodiment of the invention, the HDAC inhibitor is a stilbene like compound. In one aspect of this embodiment, the stilbene like compound is chosen from (2Z)-3-(3,5-Dimethoxy phenyl)-2-(4-fluorophenyl)-N-[6-(2-hydroxybenzylamine)-6-oxohexyllacrylamide; (2Z)-3-(3,5-Dimethoxyphenyl)-2-(4-fluorophenyl)-N-[6-(3-hydroxybenzyl amine)-6-oxohexyl]acrylamide; (2Z)-3-(3,4-Difluorophenyl)-2-(4-fluorophenyl)-N-[6-(2-thiazole amine)-6-oxo hexyl] acrylamide; (2Z)-3-(2,3,4-Trimethoxyphenyl)-2-(4-hydroxyphenyl)-N-[6-(hydroxyamino)-6-oxohexyl]acrylamide; N-hydroxy-2-{[(2Z)-3-(3,4 difluorophenyl)-2-(4-fluorophenyl)-acrylamide]6-oxohexyl]amino}-3-(4-hydroxyphenyl)propanamide, and pharmaceutically acceptable salts thereof.

In a yet further preferred embodiment of the invention, the HDAC inhibitor is a sulphonylpyrrole derivative. In one aspect of this embodiment, the sulphonylpyrrole derivative is chosen from (E)-N-Hydroxy-3-[1-(toluene-4-sulfonyl)-1-H-pyrrol-3-yl]-acrylamide; (E)-N-(2-Amino-phenyl)-3-[1-(biphenyl-4-sulfonyl)-1H-pyrrol-3-yl]-acrylamide; (E)-3-[1-(4-Aminomethyl-benzenesulfonyl)-1H-pyrrol-3-yl]-N-hydroxy-acrylamide; (E)-N-Hydroxy-3-[1-(5-pyridin-2-yl-thiophene-2-sulfonyl)-1H-pyrrol-3-yl]-acrylamide; (E)-N-(2-Amino-phenyl)-3-{1-[3-(1H-pyrazol-4-yl)-benzenesulfonyl]-1H-pyrrol-3-yl}-acrylamide, and pharmaceutically acceptable salts thereof.

In a yet further preferred embodiment of the invention, the HDAC inhibitor is a thiophene or thiazole substituted trifluoroethanone derivative. In one aspect of this embodiment, the thiophene or thiazole substituted trifluoroethanone derivative is chosen from N-(4-Methoxybenzyl)-5-(trifluoroacetyl)thiophene-2-carboxamide; N-Methyl-N-(quinolin-7-ylmethyl)-5-(trifluoroacetyl)thiophene-2-carboxamide; N-Ethyl-5-(trifluoroacetyl)thiophene-2-carboxamide; N-(4-Chlorobenzyl)-5-(trifluoroacetyl)thiophene-2-carboxamide; N-[2-(3,4-Dihydroquinolin-1(2H)-yl)ethyl]-5-(trifluoroacetyl)thiophene-2-carboxamide; 2,2,2-Trifluoro-1-(5-{[4-(3-pyridin-3-yl-1,2,4-oxadiazol-5-yl)piperidin-1-yl]carbonyl}-2-thienyl)ethanone; 1-(5-{[4-(1,3-Benzothiazol-2-yl)piperazin-1-yl]carbonyl}-2-thienyl)-2,2,2-trifluoroethanone; 1-{5-[(4-Benzoylpiperidin-1-yl)carbonyl]-2-thienyl}-2,2,2-trifluoroethanone; N-[2-(4-Phenyl-1,3-thiazol-2-yl)ethyl]-5-(trifluoroacetyl)thiophene-2-carboxamide; 1-(5-{[4-(1,3-Benzothiazol-2-yl)piperazin-1-yl]carbonyl}-2-thienyl)-2,2,2-trifluoroethanone; N-[4-(2-Chlorophenyl)-1,3-thiazol-2-yl]-5-(trifluoroacetyl)thiophene-2-carboxamide; N-[(3-chlorobenzyl)sulfonyl]-5-(trifluoroacetyl)thiophene-2-carboxamide, and pharmaceutically acceptable salts thereof.

In a yet further preferred embodiment of the invention, the HDAC inhibitor is an amino acid derivative. In one aspect of this embodiment, the amino acid derivative is chosen from N²-[(5-Methoxy-2-methyl-1H-indol-3-yl)acetyl]-N′-2-naphthyl-5-(4-oxopentyl)-Z-cystein amide; N²-[(5-Methoxy-2-methyl-1H-indol-3-yl)acetyl]-N′-2-naphthyl-5-[(2-oxopropyl)sulfonyl]-Z-norvalinamide; N-(3-Acetylphenyl)-N²-(cyanoacetyl)-5-[(3,3,3-trifluoro-2-oxopropyl)thio]-L-norvalinamide; N-Benzoylglycyl-N-pyridinium-3-yl-5-[(3,3,3-trifluoro-2-oxopropyl)thio]-L-norvalinamide trifluoroacetate; N-Benzoylglycyl-N-(3,5-dichlorophenyl)-5-[(3,3,3-trifluoro-2-oxopropyl)thio]-L-norvalinamide, and pharmaceutically acceptable salts thereof.

In a yet further embodiment of the invention, the HDAC inhibitor is a benzamide derivative (or analog). In one aspect of the invention, the benzamide derivative is chosen from N-(2-Amino-phenyl)-4-[(2-propyl-pentanoylamino)-methyl]-benzamide; N-Hydroxy-4-[(2-propyl-pentanoylamino)-methyl]-benzamide; N-(2-Amino-phenyl)-4-(2-propyl-pentanoylamino)-benzamide; N-Hydroxy-4-(2-propyl-pentanoylamino)-benzamide; 2-Propyl-pentanoic acid {4-[2-amino-phenylcarbamoyl)-methyl]-phenyl}-amide; 2-Propyl-pentanoic acid (4-hydroxycarbamoyl-methyl-phenyl)-amide; 2-Propyl-pentanoic acid {4-[2-amino-phenylcarbamoyl)-ethyl]-phenyl}-amide; 2-Propyl-pentanoic acid [4-(2-hydroxycarbamoyl-ethyl)-phenyl]-amide; 2-Propyl-pentanoic acid {4-2-(2-amino-phenylcarbamoyl)-vinyl]-phenyl}-amide; and 2-Propyl-pentanoic acid [4-(2-hydroxycarbamoyl-vinyl)-phenyl]-amide; N-(2-aminophenyl)-4-(3-chloro-5-[(3,4-dimethoxybenzyl)amino]methyl}pyridin-2-yl)benzamide; N-(2-aminophenyl)-4-(3-chloro-5-[(4-methoxybenzyl)amino]methyl }pyridin-2-yl)benzamide; N-(2-aminophenyl)-4-(3-chloro-5-[(tetrahydrofuran-2-ylmethyl)amino]methyl}pyridin-2-yl)benzamide; N-(2-aminophenyl)-4-[3-chloro-5-({[(5-methylisoxazol-3-yl)methyl]amino}methyl)pyridin-2-yllbenzamide; 5N-(2-aminophenyl)-4-[5-({[(1,5-dimethyl-1H-pyrazol-3-yl)methyl]amino}methyl)-3-methylpyridin-2-yl]benzamide; N-(2-aminophenyl)-4-(3-methyl-5-[(tetrahydrofuran-2-ylmethyl)amino]methyl}pyridin-2-yl)benzamide, and pharmaceutically acceptable salts thereof.

Examples of HDAC inhibitors for use in the invention include, but are not limited to those found in international patent applications WO2006/123121 (23 Nov. 2006), WO 2006/117549 (9 Nov. 2006), WO 2006/117548 (9 Nov. 2006), WO 2004/113336 (29 Dec. 2004), WO 2007/058628 (24 May 2007), WO 2006/101456 (28 Sep. 2006), WO 2006/101455 (28 Sep. 2006), WO 2006/101454 (28 Sep. 2006), WO 2005/040161 (6 May 2005) WO 2005/040101 (6 May 2005), WO 2007/061939 (31 May 2007), WO 2007/054776 (18 May 2007), WO 2007/039404 (12 Apr. 2007), WO 2007/039403, (12 Apr. 2007), WO 2007/029036 (15 Mar. 2007), WO 2007/029035 (15 Mar. 2007), WO 2006/129105 (7 Dec. 2006), WO 2006/097460 (21 Sep. 2006), WO 2006/097449 (21 Sep. 2006), WO 2005/108367 (17 Nov. 2005), WO 2004/067480 (12 Aug. 2004), WO 2007/084390 (26 Jul. 2007), WO 2007/071961 (28 Jun. 2007), WO 2007/071956 (28 Jun. 2007), WO 2007/067995 (14 Jun. 2007), WO 2007/067994 (14 Jun. 2007), WO 2007/061978 (31 May 2007), and WO 2005/028447 (31 Mar. 2005); and those found in US patent applications 20060199829 (Sep. 7, 2006), 20050250784 (Nov. 10, 2005), 20050234033 (Oct. 20, 2005), 20050197336 (Sep. 8, 2005), 20050137232 (Jun. 23, 2005), 20040266769 (Dec. 30, 2004), 20070225373 (Sep. 27, 2007) and 20040254220 (Dec. 16, 2004), each of which is hereby incorporated by reference in its entirety.

In one embodiment of the invention, a compound is administered to an individual in need of UCHL1 up-regulation, in an amount sufficient to inhibit AOF2 (lysine specific demethylases; LSD1) activity. In a more specific aspect of this embodiment, the AOF2 inhibiting effective amount is an amount sufficient to increase the UCHL1 mRNA by 5%, 10%, 20%, 30%, 40%, 50%, 100%, 200%, or 300% or more. In another specific aspect of this embodiment, the AOF2 inhibiting effective amount is an amount sufficient to increase the UCHL1 hydrolase activity by 5%, 10%, 20%, 30%, 40%, 50%, 100%, 200%, or 300% or more. In another specific aspect of this embodiment, the AOF2 inhibiting effective amount is an amount sufficient to increase the UCHL1 protein levels by 5%, 10%, 20%, 30%, 40%, 50%, 100%, 200%, or 300% or more. In a specific aspect of this embodiment, the individual in need of UCHL1 up-regulation is an individual suspected of having Lewy Body Dementia. In another specific aspect of this embodiment, the individual in need of UCHL1 up-regulation is an individual needing or desiring prophylaxis against cognitive decline. In another specific aspect of this embodiment, the individual in need of UCHL1 up-regulation is an individual needing or desiring a reduction in the rate of cognitive decline. Administration of the compound can reduce the rate of cognitive decline in that patient (or group of patients).

In one embodiment of the invention, the compound useful for increasing UCHL1 mRNA, UCHL1 protein, and/or UCHL1 hydrolase activity is selected from the group consisting of halo-N-propargyl-l-aminoindans, indans, indoles, methylproparylamines, 5-substituted 2,4-thiazolidinediones, alkyl and alkylbenzyl ethers of substituted hydroquinones, 1,3,4-oxadiazol-2(3H)-one derivatives, 4-(benzyloxy)benzaldehyde acetyl(2-cyanoethyl)hydrazones, N-propargylhydrazines, 4-pyrrolidino derivatives, benzazepine derivatives, 3H-quinazolin-4-one derivatives, N-acylamino aryl derivatives, N-acylamino aryl derivatives, 3-phenyl-propionamidoderivatives, 3-phenyl-acrylamido derivatives, 3-phenyl-propynamido derivatives, fluorobenzamide derivatives, 2,3-dihydro-isoindol-1-one derivatives, fluoroallylamines, pyridine-2-carboxamides, pyridine amidos, silyl alkylene amines, phthalimido derivatives, N-aryl 5-aminomethyl oxazolidine-2-one, isoquinolino derivatives, oxazolo[3,4-a]quinolin-1-ones, 2,3-dihydro-imidazo[2,1-b]benzothiazoles, 5H-furanones, 3H-dihydrofuranones, (2-benzofuranyl)-1,2,3,6-tetrahydropyridines, (2-benzofuranyl)-piperidines, 4-(2-benzofuranyl)-piperidines, 2-(5,6-dimethyl-2-benzofuranyl)-piperidines, 3,4-Dihydro-2H-pyrimido(2,1-b)benzothiazoles, thioxanthen-9-ones, a 3-N-phenylacetylamino-2,6-piperdinediones, β-(Fluoromethylene)-5-hydroxytryptophans, ethylenediamine monoamides, 1,2,3,4-tetrahydrocyclopent[b]indoles, 1,2,3,3a,4,8a-hexahydrocyclopent[B]indoles, 4-(2-benzofuranyl)-piperidines, 245 ,6-dimethyl-2-benz ofuranyl)-piperidines, arylethynylphenylcyclopropylamines, cyclopent[b]indoles, benzamides, 1,2,4-oxadiazoles, oxazolidones, 3-(aminoalkylamino)-1,2-benzisoxazoles, analogs thereof, and derivatives thereof.

In one embodiment, the invention provides a method comprising: (1) identifying a patient having Dementia with Lewy Bodies and (2) administering to said patient an amount of a pharmaceutical composition effective to increase UCHL1 activity (e.g., mRNA, protein, and/or hydrolase activity) wherein said composition comprises (I) a compound chosen from halo-N-propargyl-1-aminoindans, indans, indoles, methylproparylamines, 5-substituted 2,4-thiazolidinediones, alkyl and alkylbenzyl ether of substituted hydroquinones, 1,3,4-oxadiazol-2(3H)-one derivatives, 4-(benzyloxy)benzaldehyde acetyl(2-cyanoethyl)hydrazones, N-propargylhydrazines, 4-pyrrolidino derivatives, benzazepine derivatives, 3H-quinazolin-4-one derivatives, N-acylamino aryl derivatives, N-acylamino aryl derivatives, 3-phenyl-propionamidoderivatives, 3-phenyl-acrylamido derivatives, 3-phenyl-propynamido derivatives, fluorobenzamide derivatives, 2,3-dihydro-isoindol-1-one derivatives, fluoroallylamines, pyridine-2-carboxamides, pyridine amidos, silyl alkylene amines, phthalimido derivatives, N-aryl 5-aminomethyl oxazolidine-2-one, isoquinolino derivatives, oxazolo[3,4-a]quinolin-1-ones, 2,3-dihydro-imidazo[2,1-b]benzothiazoles, 5H-furanones, 3H-dihydrofuranones, (2-benzofuranyl)-1,2,3,6-tetrahydropyridines, (2-benzofuranyl)-piperidines, 4-(2-benzofuranyl)-piperidines, 2-(5,6-dimethyl-2-benzofuranyl)-piperidines, 3,4-Dihydro-2H-pyrimido(2,1-b)benzothiazoles, thioxanthen-9-ones, a 3-N-phenylacetylamino-2,6-piperdinediones, β-(Fluoromethylene)-5-hydroxytryptophans, ethylenediamine monoamides, 1,2,3,4-tetrahydrocyclopent[b]indoles, 1,2,3,3a,4,8a-hexahydrocyclopent[B]indoles, 4-(2-benzofuranyl)-piperidines, 2-(5,6-dimethyl-2-benzofuranyl)-piperidines, arylethynylphenylcyclopropylamines, cyclopent[b]indoles, benzamides, 1,2,4-oxadiazoles, oxazolidones, 3-(aminoalkylamino)-1,2-benzisoxazoles, analogs thereof, and derivatives thereof, and (II) a pharmaceutically acceptable excipient.

Examples of halo-N-propargyl-1-aminoindans include, but are not limited to, 4-fluoro-N-propargyl-1-aminoindan, 5-fluoro-N-propargyl-1-aminoindan, 6-fluoro-N-propargyl-1-aminoindan, optically pure enantiomers thereof, pharmaceutically acceptable salts thereof. In one aspect of this embodiment, the compound is 6-fluoro-N-propargyl-1-aminoindan. In another aspect of this embodiment, the compound is (+)-6-fluoro-N-propargyl-1-aminoindan.

Examples of indole compounds include, but are not limited to, N-methyl-N-propargyl-2-[1-methyl-5-methoxyindolyl]methylamine, N-propargyl-2-[1-methyl-5-methoxyindolyl]methylamine, N-(2-butynyl)-2-[1-methyl-5-methoxyindolyl]methylamine, N-(2-butynyl)-N-methyl-2-[1-methyl-5-methoxyindolyl]methylamine, N-(2,3-butadienyl)-2-[1-methyl-5-methoxyindolyl]methylamine, N-(2,3-butadienyl)-N-methyl-2-[1-methyl-5-methoxyindolyl]methylamine, and 5-methoxyindol-2-ylmethylamine, and pharmaceutically acceptable salts thereof.

Examples of methylpropargylamine compounds include, but are not limited to, N-(2-Butyl)-N-methylpropargylamine, N-(1-Butyl)-N-methylpropargylamine, N-(2-Propyl)-N-methylpropargylamine, N-(1-Pentyl)-N-methylpropargylamine, N-(2-Pentyl)-N-methylpropargylamine, N-(1-Heptyl)-N-methylpropargylamine, N-(2-Peptyl)-N-methylpropargylamine, N-(2-Decyl)-N-methylpropargylamine, N-(2-Dodecyl)-N-methylpropargylamine, and pharmaceutically acceptable salts thereof.

Examples of 5-substitutued 2,4-thiazolidinediones include, but are not limited to, 2,4-Dioxo-5-[3-(phenylmethoxy)-phenylmethylene]-4-thiazolidinebutanenitrile, 2,4-Dioxo-5-[3-(phenylmethoxy)-phenylmethylene]-4-thiazolidinepentanenitrile, and pharmaceutically acceptable salts thereof.

Examples of alkyl or alkylbenzyl ethers of substituted hydroquinones include, but are not limited to, 4-[2′-Formyl-4′-(m-chlorophenylmethyloxy)phenoxy]butyronitrile, 4-[2′-Methoxymethyl-4′-(m-chlorophenylmethyloxy)phenoxy]butyronitrile, 442′-Carbomethoxy-4′-(m-chlorophenylmethyloxy)phenoxy]butyronitrile, 442′-Acyl-4′-(m-chlorophenylmethyloxy)phenoxy]butyronitrile, 443′-Acyl-4′-(m-chlorophenylmethyloxy)phenoxy]butyronitrile, 4-[2′-Acetyl-4′-[(3″,5″-bis-trifluoromethylphenyl)-methyloxy]phenoxy}butyronitrile, 3-[2′-Acyl-4′-(m-trifluoromethylphenylmethyloxy)phenoxy]propanol, N-2-[3′-Acyl-4′-(m-chlorophenylmethyloxy)phenoxy]-1-methylethyl-N-methylpropargylamine, 1-[2′-Acyl-4′-(m-trifluoromethylphenylmethyloxy)phenoxy]-3-methoxy-2-propanol, S(+)-1-[2′-Acyl-4′-(m-trifluoromethylphenylmethyloxy)phenoxy]-3-methoxy-2-propanol, (S)-1-[2′-Acyl-4′-(m-trifluoromethylphenylmethyloxy)phenoxy]-3-methoxy-2-propyl acetate; (S)-1-[2′-Acyl-4′-(m-trifluoromethylphenylmethyloxy)phenoxy]-3-methoxy-2-propyl methylcarbonate, and pharmaceutically acceptable salts thereof.

Examples of 1,3,4-oxadiazol-2(3H)-one derivatives include, but are not limited to, 5-[4-(4,4,4-trifluorobutoxy)phenyl]-3-methoxyethyl-1,3,4-oxadiazol-2(3H)-one, 5-[4-(4,4,4-trifluorobutoxy)phenyl]-3-hydroxyethyl-1,3,4-oxadiazol-2(3H)-one, 5-[4-(4,4,4-trifluorobutoxy)phenyl]-3-methylthioethyl-1,3,4-oxadiazol-2(3H)-one, 5-[4-(4,4,4-trifluoro-2-butenyloxy)phenyl]-3-methoxyethyl-1,3,4-oxadiazol-2(3H)-one, 5-[4-(4,4,4-trifluoro-3(R)-hydroxybutoxy)phenyl]-3-methoxyethyl-1,3,4-oxadiazol-2(3H)-one, 5-[4-(tetrahydropyran-3-ylmethoxy)phenyl]-3-methoxyethyl-1,3,4-oxadiazol-2(3H)-one, and pharmaceutically acceptable salts thereof.

Examples of 4-(benzyloxy)benzaldehyde acetyl(2-cyanoethyl)hydrazones, include, but are not limited to, 4-(benzyloxy)benzaldehyde acetyl(2-cyanoethyl)hydrazone, 4-[(4-methylbenzyl)oxy]benzaldehyde acetyl (2-cyanoethyl)hydrazone, 4-[(4-nitrobenzyl)oxy]benzaldehyde acetyl(2-cyanoethyl)hydrazone, 4-[(4-chlorobenzyl)oxy]benzaldehyde acetyl(2-cyanoethyl)hydrazone, 4-[(4-methoxybenzyl)oxy]benzaldehyde acetyl(2-cyanoethyl)hydrazone, 4-[(2,4-dichlorobenzyl)oxy]benzaldehyde acetyl(2-cyanoethyl)hydrazone, 4-[2-chlorobenzyl)oxylbenzaldehyde acetyl(2-cyanoethyl)hydrazone, 4-(benzyloxy)benzaldehyde acetyl(2-hydroxyethyl)hydrazone, 4-(benzyloxy)benzaldehyde acetyl (2-methoxycarbonyl-ethyl)hydrazone, 4-(2-phenylethoxy)benzaldehyde acetyl(2-cyanoethyl)hydrazone, 4-(benzyloxy)benzaldehyde acetyl(2-cyanopropyl)hydrazone, 4-(benzyloxy)benzaldehyde acetyl(cyanomethyl)hydrazone, 4-(benzyloxy)benzaldehyde acetyl(3-cyanopropyl)hydrazone, 4-(benzyloxy)benzaldehyde acetylpropargylhydrazone, 4-(benzyloxy)benzaldehyde(2-cyanoethyl)(ethoxycarbonyl)hydrazone, 4-(benzyloxy)benzaldehyde(2-hydroxyethyl)(ethoxycarbonyl)hydrazone, benzaldehyde(2-cyanoethyl)(N-methylcarbamoyl)hydrazone, 4-(benzyloxy)benzaldehyde(2-cyanoethyl)(N-methylcarbamoyl)hydrazone, benzaldehyde(2-cyanoethyl)(N-phenylcarbamoyl)hydrazone, 4-(benzyloxy)benzaldehyde(2-cyanoethyl)(N-phenylcarbamoyl)hydrazone, 4-(benzyloxy)acetophenone acetyl(2-cyanoethyl)hydrazone, and pharmaceutically acceptable salts thereof.

Examples of N-propargylhydrazines include, but are not limited to, N²-propargylphenelzine, N¹-propargylphenelzine, N¹-propargyl-N²-acetylphenelzine, and pharmaceutically acceptable salts thereof.

Examples of 4-pyrrolidino derivatives include, but are not limited to, (RS)-1-[4-(3-fluoro-benzyloxy)-phenyl]-2-oxo-pyrrolidine-3-carboxylic acid methylamide, (RS)-1-[4-(3-fluoro-benzyloxy)-phenyl]-2-oxo-pyrrolidine-3-carboxylic acid amide, (RS)-1-[4-(4-fluoro-benzyloxy)-phenyl]-2-oxo-pyrrolidine-3-carboxylic acid amide, (RS)-1-[4-(4-fluoro-benzyloxy)-phenyl]-2-oxo-pyrrolidine-3-carboxylic acid methylamide, (RS)-2-oxo-1-[4-(4-trifluoromethyl-benzyloxy)-phenyl]-pyrrolidine-3-carboxylic acid amide, and (RS)-2-oxo-1-[4-(4-trifluoromethyl-benzyloxy)-phenyl] -p yrrolidine-3-carboxylic acid methylamide, (S)—N-[1-(4-benzyloxy-phenyl)-2-oxo-pyrrolidin-3-yl]-acetamide, (S)—N-[1-(4-benzyloxy-phenyl)-2-oxo-pyrrolidin-3-yl]-methanesulfonamide, (S)—N-{1-[4-(3-fluoro-benzyloxy)-phenyl]-2-oxo-pyrrolidin-3-yl}-acetamide, (R)—N-{1-[4-fluoro-benzyloxy)-phenyl]-2-oxo-pyrrolidin-3-yl}-methanesulfonamide, (S)—N-{1-[4-(3-fluoro-benzyloxy)-phenyl]-2-oxo-pyrrolidin-3-yl}-methanesulfonamide, and (S)-{1-[4-(3-fluoro-benzyloxy)-phenyl]-2-oxo-pyrrolidin-3-yl}-carbamic acid methyl ester, and pharmaceutically acceptable salts thereof.

Examples of benzazepine derivatives include, but are not limited to, 1-[7-(3-fluoro-benzyloxy)-1,2,4,5-tetrahydro-benzo[d]azepin-3-yl]-ethanone, 1-[7-(3-fluoro-benzyloxy)-1,2,4,5-tetrahydro-benzo[d]azepin-3-yl]-2-methoxy-ethanone, 2-[7-(3-fluoro-benzyloxy)-1,2,4,5-tetrahydro-benzo[d]azepin-3-yl]-2-oxo-acetamide, 3-[7-(3-fluoro-benzyloxy)-1,2,4,5-tetrahydro-benzo[d]azepin-3-yl]-3-oxo-propionamide, 7-(3-fluoro-benzyloxy)-1,2,4,5-tetrahydro-benzo[d]azepine-3-carboxylic acid methyl ester, 7-(3-fluoro-benzyloxy)-1,2,4,5-tetrahydro-benzo[d]azepine-3-carbaldehyde, 7-(3-fluoro-benzyloxy)-3-methanesulfonyl-2,3,4,5-tetrahydro-1H-benzo[d]azepine, 7-(3-fluoro-benzyloxy)-1,2,4,5-tetrahydro-benzo[d]azepine-3-carboxylic acid amide, 7-(3-fluoro-benzyloxy)-1,2,4,5-tetrahydro-benzo[d]azepine-3-carboxylic acid ethylamide, 2-[7-(3-fluoro-benzyloxy)-1,2,4,5-tetrahydro-benzo [d]azepin-3-yl]-acetamide, (RS)-2-[7-(3-fluoro-benzyloxy)-1,2,4,5-tetrahydro-benzo[d]azepin-3-yl]-propionamide, and pharmaceutically acceptable salts thereof.

Examples of 3H-quinazolin-4-one derivatives, include, but are not limited to, 2-[7-(3-fluoro-benzyloxy)-4-oxo-4H-quinazolin-3-yl]-acetamide, 2-[7-(3-fluoro-benzyloxy)-4-oxo-4H-quinazolin-3-yl]-propionamide, 2-[7-(4-fluoro-benzyloxy)-4-oxo-4H-quinazolin-3-yl]-acetamide, 2-[7-(4-fluoro-benzyloxy)-4-oxo-4H-quinazolin-3-yl]-propionamide, 2-[7-(3-fluoro-benzyloxy)-2-methyl-4-oxo-4H-quinazolin-3-yl]-acetamide, 2-[2-cyclopropyl-7-(3-fluoro-benzyloxy)-4-oxo-4H-quinazolin-3-yl]-acetamide, 7-(3-fluoro-benzyloxy)-3-(2-methoxy-ethyl)-3H-quinazolin-4-one, 7-(4-fluoro-benzyloxy)-3-(2-methoxy-ethyl)-3H-quinazolin-4-one, 7-(3-fluoro-benzyloxy)-3-(2-methoxy-ethyl)-2-methyl-3H-quinaz olin-4-one, 3-(2-amino-ethyl)-7-(3-fluoro-benzyloxy)-3H-quinazolin-4-one, 3-(3-amino-propyl)-7-(3-fluoro-benzyloxy)-3H-quinazolin-4-one, 3-(2-amino-ethyl)-7-(4-fluoro-benzyloxy)-3H-quinazolin-4-one, 2-[7-(3-fluoro-benzyloxy)-2-methyl-4-oxo-4H-quinazolin-3-yl]-ethyl-ammonium chloride, [7-(3-fluoro-benzyloxy)-4-oxo-4H-quinazolin-3-yl]-acetic acid ethyl ester, fluoro-[7-(3-fluoro-benzyloxy)-4-oxo-4H-quinazolin-3-yl]-acetic acid ethyl ester, 2-[7-(3-fluoro-benzyloxy)-4-oxo-4H-quinazolin-3-yl]-propionic acid ethyl ester, [7-(3-fluoro-benzyloxy)-4-oxo-4H-quinazolin-3-yl]-acetic acid tert-butyl ester, 2-[7-(3-fluoro-benzyloxy)-4-oxo-4H-quinazolin-3-yl]-propionic acid tert-butyl ester, [7-(4-fluoro-benzyloxy)-4-oxo-4H-quinazolin-3-yl]-acetic acid ethyl ester, 2-[7-(4-fluoro-benzyloxy)-4-oxo-4H-quinazolin-3-yl]-propionic acid ethyl ester, and pharmaceutically acceptable salts thereof.

Examples of N-acylamino aryl derivatives include, but are not limited to, N-[4-(3-fluoro-benzyloxy)-phenyll-malonamide, N-[4-(3-fluoro-benzyloxy)-phenyl]-malonamic acid methyl ester, N-[4-(3-fluoro-benzyloxy)-phenyl]-malonamic acid methyl ester, N-[3-fluoro-4-(3-fluoro-benzyloxy)-phenyl-[malonamic acid methyl ester, N-[4-(4-fluoro-benzyloxy)-phenyl]-malonamic acid methyl ester, N-[2-fluoro-4-(3-fluoro-benzyloxy)-phenyl]-malonamic acid methyl ester, N-[4-(2,4-difluoro-benzyloxy)-phenyl]-malonamic acid methyl ester, N-[4-(2-fluoro-benzyloxy)-phenyl]-malonamic acid methyl ester, N-[4-(2,4,5-trifluoro-benzyloxy)-phenyl]-malonamic acid methyl ester, N-[2-fluoro-4-(4-fluoro-benzyloxy)-phenyl]-malonamic acid methyl ester, N-[4-(3,5-bis-trifluoromethyl-benzyloxy)-2-fluoro-phenyl]-malonamic acid methyl ester, N-[4-(3-fluoro-benzyloxy)-3-methyl-phenyl]-malonamic acid methyl ester, N-[3-chloro-4-(3-fluoro-benzyloxy)-phenyl]-malonamic acid methyl ester, cyclopropane-1,1-dicarboxylic acid amide [4-(3-fluoro-benzyloxy)-phenyl]-amide, N-[4-(3-fluoro-benzyloxy)-phenyl]-malonamide, N-[4-(3-fluoro-benzyloxy)-phenyl]-2-methyl-malonamide, N-[3-fluoro-4-(3-fluoro-benzyloxy)-phenyl]-malonamide, N-[4-(4-fluoro-benzyloxy)-phenyl]-malonamide, N-[4-(2,4-difluoro-benzyloxy)-phenyl]-malonamide, N-[4-(2,4,5-trifluoro-benzyloxy)-phenyl]-malonamide, N-[4-(2-fluoro-benzyloxy)-phenyl]-malonamide, N-(4-benzyloxy-phenyl)-malonamide, N-[4-(4-chloro-benzyloxy)-phenyl]-malonamide, N-[4-(3-fluoro-benzyloxy)-2-hydroxy-phenyl]-malonamide, N-[2-fluoro-4-(4-fluoro-benzyloxy)-phenyl]-malonamide, N-[4-(3-fluoro-benzyloxy)-3-methyl-phenyl]-malonamide, N-[3-chloro-4-(3-fluoro-benzyloxy)-phenyl]-malonamide, cyclopropane-1,1-dicarboxylic acid amide [2-fluoro-4-(4-fluoro-benzyloxy)-phenyl]-amide, 2-Acetylamino-N-[2-fluoro-4-(4-fluoro-benzyloxy)-phenyl]-acetamide, 2-Acetylamino-N-[2-fluoro-4-(3-fluoro-benzyloxy)-phenyl]-acetamide, N-[2-Fluoro-4-(4-fluoro-benzyloxy)-phenyl] -2-formylamino-acetamide, and N-[2-Fluoro-4-(3-fluoro-benzyloxy)-phenyl]-2-formylamino-acetamide, and pharmaceutically acceptable salts thereof.

Examples of 3-phenyl-propionamidos, 3-phenyl-acrylamidos, or 3-phenyl-propynamidos include, but are not limited to, N-methyl-3-[4-(4-methyl-benzyloxy)-phenyl]-acrylamide, 3-[4-(3-methoxy-benzyloxy)-phenyl]-N-methyl-acrylamide, 3-[4-(3-fluoro-benzyloxy)-phenyl]-2,N-dimethyl-acrylamide, 3-[4-(3-fluoro-benzyloxy)-phenyl]-N-methyl-acrylamide, N-methyl-3-[4-(4-trifluoromethyl-benzyloxy)-phenyl]-acrylamide, 3-[4-(3,4-difluoro-benzyloxy)-phenyl]-N-methyl-acrylamide, 3-[4-(4-fluoro-benzyloxy)-phenyl]-N-methyl-acrylamide, and pharmaceutically acceptable salts thereof.

Examples of fluorobenzamide derivatives include, but are not limited to, (S)—N-(1-carbamoyl-ethyl)-2-fluoro-4-(3-fluoro-benzyloxy)-benzamide, 2-[4-(3-fluorobenzyloxy)-2-fluoro-benzamido]acetamide, (S)—N-(1-carbamoyl-2-hydroxy-ethyl)-2-fluoro-4-(3-fluoro-benzyloxy)-benzamide, (R)—N-(1-carbamoyl-ethyl)-2-fluoro-4-(3-fluoro-benzyloxy)-benzamide, 2-[4-(4-fluorobenzyloxy)-2-fluoro-benzamido]acetamide, (S)—N-(1-carbamoyl-ethyl)-2-fluoro-4-(4-fluoro-benzyloxy)-benzamide, (S)—N-(1-carbamoyl-ethyl)-2-fluoro-4-(4-trifluoromethyl-benzyloxy)-benzamide, (S)-4-(3,5-bis-trifluoromethyl-benzyloxy)-N-(1-carbamoyl-ethyl)-2-fluoro-benzamide, (S)—N-(1-Carbamoyl-ethyl)-3-fluoro-4-(4-trifluoromethyl-benzyloxy)-benzamide, (R)—N-(1-Carbamoyl-ethyl)-3-fluoro-4-(4-trifluoromethyl-benzyloxy)-benzamide, N-Cyanomethyl-3-fluoro-4-(4-trifluoromethyl-benzyloxy)-benzamide, N-(2-Amino-ethyl)-3-fluoro -4-(4trifluoromethyl-b enzyloxy)-benzamide 1:1 hydrochloride; (R)—N-(1-Carbamoyl-ethyl)-2,6-difluoro-4-(4-fluoro-benzyloxy)-benzamide (S)—N-(1-Carbamoyl-2-hydroxy-ethyl)-2,6difluoro-4-(4-fluoro-benzyloxy)-benzamide, N-(2-Amino-ethyl)-2,6-difluoro-4-(4-fluoro-benzyloxy)-benzamide 1:1 hydrochloride, 2,6-Difluoro-4-(4-fluoro-benzyloxy)-N-(2-hydroxy-ethyl)-benzamide, (R)-2,6-Difluoro-4-(4-fluoro-benzyloxy)-N-(2-hydroxy-1-methyl-ethyl)-benzamide, (S)—N-(1-Carbamoyl-ethyl)-2,6-difluoro-4-(3-fluoro-benzyloxy)-benzamide, (R)—N-(1-Carbamoyl-ethyl)-2,6-fluoro-4-(3-fluoro-benzyloxy)-benzamide, and pharmaceutically acceptable salts thereof.

Examples of a 2,3-Dihydro-isoindol-1-one derivatives include, but are not limited to, 2-[5-(3-fluoro-benzyloxy)-1-oxo-1,3-dihydro-isoindol-2-yl]-acetamide, 2-[5-(3-fluoro-benzyloxy)-1-oxo-1,3-dihydro-isoindol-2-yl]-propionamide, (S)-2-[6-(3-fluoro-benzyloxy)-1-oxo-1,3-dihydro-isoindol-2-yl]-propionamide, (R)-2-[6-(3-fluoro-benzyloxy)-1-oxo-1,3-dihydro-isoindol-2-yl]-propionamide, (S)-2-[1-oxo-6-(4-trifluoromethyl-benzyloxy)-1,3-dihydro-isoindol-2-yl]-propionamide, (R)-2-[1-oxo-6-(4-trifluoromethyl-benzyloxy)-1,3-dihydro-isoindol-2-yl]-propionamide, [-[6-(3-fluoro-benzyloxy)-1-oxo-1,3-dihydro-isoindol-2-yl]-acetamide, (R)-2-[6-(3-fluoro-benzyloxy)-1-oxo-1,3-dihydro-isoindol-2-yl]-propionamide, (S)-2-[1-oxo-6-(4-trifluoromethyl-benzyloxy)-1,3-dihydro-isoindol-2-yl]-propionamide, (R)-2-[1-oxo-6-(4-trifluoromethyl-benzyloxy)-1,3-dihydro-isoindol-2-yl]-propionamide, 2-(2-Methoxy-ethyl)-6-(3-fluoro-benzyloxy)-2,3-dihydro-isoindol-1-one, 2-(2-methoxy-ethyl)-6-(4-trifluoromethyl-benzyloxy)-2,3-dihydro-isoindol-1-one, 2-(2-amino-ethyl)-6-(4-trifluoromethyl-benzyloxy)-2,3-dihydro-isoindol-1-one 1:1 hydrochloride, 2-(2-amino-ethyl)-6-(4-trifluoromethyl-benzyloxy)-2,3-dihydro-isoindol-1-one 1:1 hydrochloride, and pharmaceutically acceptable salts thereof.

Examples of fluoroallylamines include, but are not limited to, 2-isobutyl-3-fluoroallylamine, 2-isopropyl-3-fluoroallylamine, 2-(9-octadecenyl)-3-fluoroallylamine, 2-(3-methyl-3-butenyl)-3-fluorallylamine, 2-(4-methoxy-2-butenyl)-3-fluoroallylamine, 2-isobutylsulfonylmethyl-3-fluoroallylamine, 2-sec-butyl-3-fluoroallylamine, 2-fluoroallylamine, 2-hexyl-3-fluoroallylamine, 2-heptyl-3-fluoroallylamine, 2-ethoxymethyl-3-fluoroallylamine, and 2-thioethoxymethyl-3-fluoroallylamine, 2-(2′-chlorophenoxy)methyl-3-fluoroallylamine, 2-(4′-chlorophenoxy)methyl-3-fluoroallylamine, 2-(4′-fluorophenoxy)methyl-3-fluoroallylamine, 2-thiophenoxymethyl-3-fluoroallylamine, 2-(2′,4′-dichlorophenoxy)methyl-3-fluoroallylamine, 2-(2′,4′-dichlorothiophenoxy)methyl-3-fluoroallylamine, 2-(5′-chloro-3′-fluorophenoxy)methyl-3-fluoroallylamine, 2-(2′chlorothiophenoxy)methyl-3-fluoroallylamine, 2-(4′-fluorothiophenoxy)methyl-3-fluoroallylamine, 2-phenoxymethyl-3-fluoroallylamine, and 2-(2′-chloro-4′-fluorothiophenoxy)methyl-3-fluoroallylamine, and pharmaceutically acceptable salts thereof.

Examples of a pyridine-2-carboxamides include but are not limited to, N-(2-aminoethyl)-5-chloropyridine-2-carboxamide and pharmaceutically acceptable salts thereof.

Examples of silyl alkylene amines include, but are not limited to, β-(benzyldimethylsilyl)ethanamine.hydrochloride, β-(dimethyl-2-phenylethylsilyl)ethanamine.hydrochloride, ethyl-4-fluorobenzylmethylsilylmethanamine.hydrochloride, dimethyl-4-fluorobenzylsilylmethanamine.hydrochloride, dimethyl-3-fluorobenzylsilylmethanamine.hydrochloride, 3,4-difluorobenzyldimethylsilylmethanamine.hydrochloride, 2,6-difluorobenzyldimethylsilylmethanamine, hydrochloride, 2,4-difluorobenzyl)dimethylsilylmethanamine.hydrochloride, dimethyl-2-fluorobenzylsilylmethanamine.hydrochloride, cyclohexylmethyldimethylsilylmethanamine.hydrochloride, β-(benzyldimethylsilyl)ethanamine.hydrochloride, β-(dimethyl-2-phenylethylsilyl)ethanamine.hydrochloride, ethyl-4-fluorobenzylsilylmethanamine.hydrochloride, dimethyl-4-fluorobenzylsilylmethanamine.hydrochloride, dimethyl-3-fluoroenzylsilylmethanamine.hydrochloride, 3,4-difluorobenzyldimethylsilylmethanamine.hydrochloride, 2,6-difluorobenzyldimethylsilylmethanamine.hydrochloride, 2,4-difluorobenzyldimethylsilylmethanamine.hydrochloride, dimethyl-2-fluoroenzylsilylmethanamine.hydrochloride, andcyclohexylmethyldimethylsilylmethanamine.hydrochloride, ethyl-4-fluorobenzylmethylsilylmethanamine.hydrochloride, dimethyl-4-fluorobenzylsilylmethanamine.hydrochloride, dimethyl-3-fluorobenzylsilylmethanamine.hydrochloride, 3,4-difluorobenzyldimethylsilylmethanamine.hydrochloride, 2,6-difluorobenzyldimethylsilylmethanamine, hydrochloride, 2,4-difluorobenzyl)dimethylsilylmethanamine.hydrochloride, dimethyl-2-fluorobenzylsilylmethanamine.hydrochloride, and cyclohexylmethyldimethylsilylmethanamine.hydrochloride.

Examples of phthalimido derivatives include, but are not limited to, 2-[5-(4-fluoro-benzyloxy)-1,3-dioxo-1,3-dihydro-isoindol-2-yl]-acetamide, (S)-2-[5-(4-fluoro-benzyloxy)-1,3-dioxo-1,3-dihydro-isoindol-2-yl]-propionamide, (S)-2-[5-(4-fluoro-benzyloxy)-1,3-dioxo-1,3-dihydro-isoindol-2-yl]-3-hydroxy-propionamide, (R)-2-[5-(4-fluoro-benzyloxy)-1,3-dioxo-1,3-dihydro-isoindol-2-yl]-propionamide, 2-[5-(3-fluoro-benzyloxy)-1,3-dioxo-1,3-dihydro-isoindol-2-yl]-propionamide, (2-[5-(3-fluoro-benzyloxy)-1,3-dioxo-1,3-dihydro-isoindol-2-yl]-acetamide, 2-[5-(3-fluoro-benzyloxy)-1,3-dioxo-1,3-dihydro-isoindol-2-yl]-3-hydroxy-propionamide, N-{2-[5-(4-fluoro-benzyloxy)-1,3-dioxo-1,3-dihydro-isoindol-2-yl]-ethyl}-acetamide, 2-(2-amino-ethyl)-5-(4-fluoro-benzyloxy)-isoindole-1,3-dione, 5-(4-fluoro-benzyloxy)-2-piperidin-4-yl-isoindole-1,3-dione, 5-(4-fluoro-benzyloxy)-2-(2-hydroxy-ethyl)-isoindole-1,3-dione, 5-(4-fluoro-benzyloxy)-2-(2-methoxy-ethyl)-isoindole-1,3-dione, 5-(3-fluoro-benzyloxy)-2-(2-methoxy-ethyl)-isoindole-1,3-dione, (S)-5-(4-fluoro-benzyloxy)-2-(2-methoxy-1-methyl-ethyl)-isoindole-1,3-dione, (S)-5-(3-fluoro-benzyloxy)-2-(2-methoxy-1-methyl-ethyl)-isoindole-1,3-dione, (S)-5-(2-fluoro-benzyloxy)-2-(2-methoxy-1-methyl-ethyl)-isoindole-1,3-dione, (S)-2-(2-methoxy-1-methyl-ethyl)-5-(4-trifluoromethyl-benzyloxy)-isoindole-1,3-dione, (S)-5-(4-bromo-benzyloxy)-2-(2-methoxy-1-methyl-ethyl)-isoindole-1,3-dione, (S)-5-(3,4-difluoro-benzyloxy)-2-(2-methoxy-1-methyl-ethyl)-isoindole-1,3-dione, 5-(3-fluoro-benzyloxy)-2-(2-hydroxy-ethyl)-isoindole-1,3-dione, 5-(4-fluoro-benzyloxy)-2-(3,3,3-trifluoro-2-hydroxy-propyl)-isoindole-1,3-dione, 5-(3,5-bis-trifluoromethyl-benzyloxy)-2-(2-methoxy-1-methyl-ethyl)-isoindole-1,3-dione, and pharmaceutically acceptable salts thereof.

Examples of isoquinolino derivatives include, but are not limited to, 2-[6-(3-fluoro-benzyloxy)-1-oxo-3,4-dihydro-1H-isoquinolin-2-yl]-acetamide, 2-[6-(3-fluoro-benzyloxy)-1-oxo-3,4-dihydro-1H-isoquinolin-2-yl]-propionamide 2-[6-(4-fluoro-benzyloxy)-1-oxo-3,4-dihydro-1H-isoquinolin-2-yl]-propionamide, 2-[6-(3,4-difluoro-benzyloxy)-1-oxo-3,4-dihydro-1H-isoquinolin-2-yl]-propionamide, 2-[6-(3-fluoro-benzyloxy)-1-oxo-3,4-dihydro-1H-isoquinolin-2-yl]-propionamide, 2-(R)-[6-(3-fluoro-benzyloxy)-1-oxo-3,4-dihydro-1H-isoquinolin-2-yl]-propionamide, 2-(R)-[6-(4-fluoro-benzyloxy)-1-oxo-3,4-dihydro-1H-isoquinolin-2-yl]-propionamide, 2-(S)-[6-(4-fluoro-benzyloxy)-1-oxo-3,4-dihydro-1H-isoquinolin-2-yl]-propionamide, 2-(S)46-(4-fluoro-benzyloxy)-1-oxo-3,4-dihydro-1H-isoquinolin-2-yl]-3-hydroxy-propionamide, 2-(R)-[6-(2,6-difluoro-benzyloxy)-1-oxo-3,4-dihydro-1H-isoquinolin-2-yl]-propionamide, 2-[6-(3-fluoro-benzyloxy)3,4-dihydro-1H-isoquinolin-2-yl]-propionamide, 2-[6-(4-fluoro-benzyloxy)3,4-dihydro-1H-isoquinolin-2-yl]-acetamide, 2-[6-(3-fluoro-benzyloxy)-3,4-dihydro-1H-isoquinolin-2-yl]-acetamide, 2-[6-(4-fluoro-benzyloxy)3,4-dihydro-1H-isoquinolin-2-yl]-propionamide, 2-(R)-[6-(4-fluoro-benzyloxy)-1,3-dioxo-3,4-dihydro-1H-isoquinolin-2-yl]-propionamide, 2-(S)-[6-(4-fluoro-benzyloxy)-1,3-dioxo-3,4-dihydro-1H-isoquinolin-2-yl]-propionamide, 2-(S)-[6-(4-fluoro-benzyloxy)-3-oxo-3,4-dihydro-1H-isoquinolin-2-yl]-propionamide, 2(R)-[6-(4-fluoro-benzyloxy)-3-oxo-3 ,4-dihydro-1H-isoquinolin-2-yl]-propionamide, and pharmaceutically acceptable salts thereof.

Examples of pyridine amidos include but are not limited to 5-(3-fluoro-benzyloxy)-pyridine-2-carboxylic acid carbamoylmethyl-amide, 5-(4-fluoro-benzyloxy)-pyridine-2-carboxylic acid carbamoylmethyl-amide, 5-(3,4-difluoro-benzyloxy)-pyridine-2-carboxylic acid carbamoylmethyl-amide, (S)-5-(3-fluoro-benzyloxy)-pyridine-2-carboxylic acid (1-carbamoyl-ethyl)-amide, (S)-5-(4-fluoro-benzyloxy)-pyridine-2-carboxylic acid (1-carbamoyl-ethyl)-amide, (S)-5-(3,4-difluoro-benzyloxy)-pyridine-2-carboxylic acid (1-carbamoyl-ethyl)-amide, 6-Benzyloxy-N-carbamoylmethyl-nicotinamide, N-Carbamoylmethyl-6-(3-fluoro-benzyloxy)-nicotinamide, N-Carbamoylmethyl-6-(4-fluoro-benzyloxy)-nicotinamide, (S)-6-B enzyloxy-N-(1-carbamoyl-ethyl)-nicotinamide, (S)—N-(1-Carbamoyl-ethyl)-6-(3-fluoro-benzyloxy)-nicotinamide, and (S)—N-(1-Carbamoyl-ethyl)-6-(4-fluoro-benzyloxy)-nicotinamide, and pharmaceutically acceptable salts thereof.

Examples of oxazolo[3,4-a]quinolin-1-ones, include, but are not limited to, 3-methoxymethyl-7-(4,4,4-trifluoro-3-hydroxybutoxy)-3,3a,4,5-tetrahydro-1H-oxazolo [3,4-a]quinolin-1-one, 3-methoxymethyl-7-(4,4,4-trifluorobutoxy)-3,3a,4,5-tetrahydro-1H-oxazolo[3,4-a]quinolin-1-one, 7-(4,4,4-trifluorobutoxy)-3,3a,4,5-tetrahydro-1H-oxazolo[3,4-a]quinolin-1-one, 7-(3-hydroxy-4,4,4-trifluorobutoxy)-3,3a,4 ,5-tetrahydro-1H-oxazolo[3,4-a]quinolin-1-one, 3-methoxymethyl-7-[(2-(1-hydroxycyclopenyl)ethoxy]-3,3a,4,5-tetrahydro-1H-oxazolo[3,4-a]quinolin-1-one.

Examples of a 3,4-Dihydro-2H-pyrimido(2,1-b)benzothiazoles include, but are not limited to, N-(1-ethylpropyl)-3,4-dihydro-2H-pyrimido[2,1-b]benzothiazol-7-amine, the pharmaceutically acceptable acid addition salts thereof, and the pyrimido[2,1-b]benzothiazolium salts thereof.

Examples of thioxanthen-9-ones, include, but are not limited to, 7-isopropyl-3-(2-methyl)-2H-tetrazol-5-yl)thioxanthen-9-one 10,10-dioxide, 3-(1-methyl-1H-tetrazol-5-yl)thioxanthen-9-one 10,10-dioxide, and 3-(1-methyl-1H-tetrazol-5yl)thioxanthen-9-one 10,10-dioxide, and pharmaceutically acceptable salts thereof.

Examples of such ethylenediamine monoamides include, but are not limited to, N-(2-aminoethyl)-4-methoxypyridine-2-carboxamide, N-(2-aminoethyl)thiazole-2-carboxamide, N-(2-aminoethyl)-4-bromopyridine-2-carboxamide, N-(2-aminoethyl)-4-chloropyridine-2-carboxamide, N-(2-aminoethyl)-2-chlorothiazole-4-carboxamide, N-(2-aminoethyl)-5-methylisoxazole-3-carboxamide, N-(2-aminoethyl)-6-bromopyridine-2-carboxamide, N-(2-aminoethyl)-6-chloropyridine-2-carboxamide, N-(2-aminoethyl)-5-bromothiazole-4-carboxamide, N-(2-aminoethyl)-3-aminopyridine-2-carboxamide, N-(2-aminoethyl)pyridine-2-carboxamide, N-(2-aminoethyl)-5-chloropyridine-2-carboxamide, and pharmaceutically acceptable salts thereof.

Examples of 1,2,3,4-tetrahydrocyclopent[b]indoles and 1,2,3,3a,4,8a-hexahydrocyclopent[B]indoles include, but are not limited to, 4-methyl-3-phenylmethylamino-1,2,3,4-tetrahydrocyclopent[b]indol7-yl methylcarbamate, 3-(N-cyclopropyl)amino-4-methyl-1,2,3,4-tetrahydrocyclopent[b]indol7-yl methylcarbamate, 1,2,3,3a,4,8-hexahydro-4-methyl-3-(N-phenylmethyloxycarbonyl)amino-cyclopent[b]indol-7-yl methylcarbamate, 1,2,3,3a,4,8a-hexahydro-4-methyl-3-(N-phenylmethyl-N-methylaminocarbonyl)aminocyclopent[b]indol-7-yl phenylmethylcarbamate, 4-methyl-3-(2-phenylethyl)amino-1,2,3,4-tetrahydrocyclopent[b]indol7-yl methylcarbamate, 4-methyl-3-(2-phenylethyl)amino-1,2,3,4-tetrahydrocyclopent[b]indol7-yl benzylcarbamate, and pharmaceutically acceptable salts thereof.

Examples of arylethynylphenylcyclopropylamines include, but are not limited to, 1-[4-(biphenylylethynyl)phenyl]cyclopropylamine; 1-[4-(p-tolylethynyl)phenyl]cyclopropylamine; 1-[4-(2-methoxyphenylethynyl)phenyl]cyclopropyl-N-methylamine; 1-[4-(3-fluorophenylethynyl)phenyl]cyclopropylmorpholine; 1-[4-(phenylethynyl)phenyl]cyclopropyl-N,N-di-t-butylamine; 1-[4-(3-iodophenylethynyl)phenyl]-cyclopropylamine; 1-[4-(3,5-dibromophenylethynyl)phenyl]cyclopropyl-N-propylamine; 1-[4-(4-cyclohexylphenylethynyl)phenyl]cyclopropyl-N-cyclopropylamine; 1-[4-(phenylethynyl)phenyl]cyclopropyl-N-hexylamine and pharmaceutically acceptable salts thereof

Examples of cyclopent[b]indoles include, but are not limited to, 4-Methyl-3-(2propynyl)amino-1,2,3,4-tetrahydrocyclopent[b]-indol-7-yl-methylcarbamate,

1,2,3,3a,4,8b-Hexahydro-4-methyl-3-(N-phenylmethoxycarbonyl)aminocyclopent[b]indol-7-yl methylcarbamate, 1,2,3,3a,4,8b-Hexahydro-4-methyl-3-(N-phenylmethyl-N-methylaminocarbonyl)aminocyclopent[b]indol-7-yl-phenylmethylc arb amate, and pharmaceutically acceptable salts thereof.

Examples of benzamides include, but are not limited to, N-(2-Aminoethyl)-p-chlorobenzamide, N-(2-aminoethyl)-p-fluorobenzamide, N-(2-aminoethyl)-p-bromobenzamide, N-(2-aminoethyl)-3,4-dichlorobenzamide, N-(2-aminoethyl)-2,4-dichlorobenzamide and N-(2-aminoethyl)benzamide, and pharmaceutically acceptable salts thereof.

Examples of 1,2,4-oxadiazoles include, but are not limited to, 3-[4-[3-(1H-imidazol-1yl)propoxy]phenyl]-5-ethyl-1,2,4-oxadiazole, 3-[4-[3-(1H-imidazol-1-yl)propoxy]phenyl]-5-trichloromethyl-1,2,4-oxadiazole, 3-[4-[3-(1H-imidazol-1-yl)propoxy]phenyl]-5-propyl-1,2,4-oxadiazole, 3-[4-[3-(1H-imidazol-1-yl)propoxy]phenyl]-5-cyclopropyl-1,2,4-oxadiazole, 3-[4-[3-(1H-imidazol-1-yl)propoxy]phenyl]-5-phenyl-1,2,4-oxadiazole, 3-[4-[3-(3-pyridyl)propoxy]phenyl]-5-ethyl-1,2,4-oxadiazole, 3-[4-[2-(1H-imidazol-1-yl)ethoxy]phenyl]-5-ethyl-1,2,4-oxadiazole, 3-(4-benzyloxy)phenyl-5-ethyl-1,2,4-oxadiazole, 3-(4-benzyloxy)phenyl-5-trichloromethyl-1,2,4-oxadiazole, 3-[4-[3-(1H-imidazol-1-yl)propoxy]phenyl]-5-trifluoromethyl-1,2,4-oxadiazole, 3-[4-[3-(1H-imidazol-1-yl)propoxy]phenyl]-5-pentafluoroethyl-1,2,4oxadiazole, 3-[4-[3-(1H-imidazol-1-yl)propoxy]phenyl]-5-heptafluoropropyl-1,2,4-oxadiazole, 3-[4-[3-(1H-imidazol-1-yl)propoxy]phenyl]-5-methyl-1,2,4-oxadiazole, 3-[4-(3pyridylmethyloxy)phenyl]-5-methyl-1,2,4-oxadiazole, 3-[4-(4pyridylmethyloxy)phenyl]-5-methyl-1,2,4-oxadiazole, 3-[4-(3phenylpropoxy)phenyl]-5-methyl-1,2,4-oxadiazole, 3-(4-benzyloxy)phenyl]-5-methyl-1,2,4-oxadiazole, 3-[4-(3-chlorobenzyloxy)phenyl]-5-methyl-1,2,4-oxadiazole, 3-[4-[3-(1H-imidazol-1-yl)propoxy]phenyl]-5-methylamino-1,2,4-oxadiazole and 3-(4-benzyloxyphenyl)-5-methylamino-1,2,4-oxadiazole, and pharmaceutically acceptable salts thereof.

Examples of oxazolidones include, but are not limited to, 3-[2-(1-hydroxy-3-cyanopropyl)benzothiazol-6-yl]-5-methoxymethyl-2-oxazolidone; 3-[2-(1(S)-hydroxy-3-cyanopropyl)benzothiazol-6-yl]-5(R)-methoxymethyl-2-oxazolidone; 3-[2-(1(R)-hydroxy-3-cyanopropyl)benzothiazol-6-yl]-5(R)-methoxymethyl-2-oxazolidone; 3-[2-(3-cyanopropyl)benzothiazol-6-yl]-5-methoxymethyl-2-oxazolidone; 3-[2-(3-cyanopropyl)benzothiazol-6-yl]-5(R)-methoxymethyl-2-oxazolidone; 3-[2-(3-cyanopropyl)benzothiazol-6-yl]-5(S)-methoxymethyl-2-oxazolidone; 3-[2-(1-hydroxy-4-cyanobutyl)benzothiazol-6-yl]-5-methoxymethyl-2-oxazolidone; 3-[2-(4-cyanobutyl)benzothiazol-6-yl]-5-methoxymethyl-2-oxazolidone; 3-[2-(4-cyanobutyl)benzothiazol-6-yl]-5(S)-methoxymethyl-2-oxazolidone; 3-[2-(4-cyanobutyl)benzothiazol-6-yl]-5(R)-methoxymethyl-2-oxazolidone; 3-[2-(2-cyanoethyl)benzothiazol-6-yl]-5-methoxymethyl-2-oxazolidone; 3-[2-(3-cyanopropyl)benzothiazol-6-yl]-5(S)-hydroxymethyl-2-oxazolidone; 3-[2-(3-cyanopropyl)benzothiazol-6-yl]-5(R)-hydroxymethyl-2-oxazolidone; 3-[2-(3-(cyano)-1-propenyl)benzothiazol-6-yl]-5-methoxymethyl-2-oxazolidone; 3-[2-(4-cyanobutyl)benzothiazol-6-yl]-5(S)-hydroxymethyl-2-oxazolidone; 3-[2-(3-(cyano)-1,1-dimethylpropyl)benzothiazol-6-yl]-5(R)-methoxymethyl-2-oxazolidone; 3-[2-(3-(cyano)-1,1-dimethylpropyl)benzothiazol-6-yl]-5(R)hydroxymethyl-2-oxazolidone; 3-[2-(3-(cyano)-1(R)-hydroxy, 1(S)methyl-propy)benzothiazol-6-yl]-5(R)-methoxymethyl-2-oxazolidone; 3-[2-(3-(cyano)-1(S)-hydroxy, 1(R)methyl-propyl)benzothiazol-6-yl]-5(R)-methoxymethyl-2-oxazolidone, and pharmaceutically acceptable salts thereof.

Examples of 3-(aminoalkylamino)-1,2-benzisoxazoles include, but are not limited to, 6-Methoxy-N-methyl-N-[2-(4-morpholinyl)ethyl]-1,2-benzisoxazol-3-amine; 3-[[2-(4-Morpholinyl)-ethyl]methylamino]-1,2-benzisoxazol-6-ol; 3-[[2-(4-Morpholinyl)-ethyl]methylamino]-1,2-benzisoxazol-6-yl methylcarbamate; 3-[[2-(4-Morpholin-yl)ethyl]methylamino]-1,2-benzisoxazol-6-ylphenylmethyl carbamate; 3-[[2-(4-Morpholinyl)-ethyl]methylamino]-1,2-benzisoxazol-6-yl-1-methylethyl-carbamate; N-methyl-N-[2-(4-morpholinyl)ethyl]-1,2-benzisoxazol-3-amine; N-[2-(4-Morpholinyl)ethyl]-1,2-benzisoxazol-3-amine; 6-Methoxy-N42-(4-morpholinyl)ethyl]-1,2-benzisoxazol-3-amine; 3-[[2-(4-Morpholinyl)ethyl]amino]-1,2-benzisoxazol-6-ol; 3-[[2-(4-Morpholinyl)-ethyl]methylamino1-1,2-benzisoxazol-5-ol; 3-[[2-(4-Morpholinyl)-ethyl]amino]-1,2-benzisoxazol-6-y1 methylcarbamate; 3-[[2-(4-Morpholinyl)ethyl]amino]-1,2-benzisoxazol-5-y1 methylcarbamate; 6-Chloro-N-[2-(4-morpholinyl)ethyl]-1,2-benzisoxazol-3-amine; 1-Methyl-N-[2-(4-morpholinyl)ethyl]-1,2-indazol-3-amine; N-Methyl-N42-(4-morpholinyl)ethyl]-1,2-benzisothiazol-3-amine; 5-Methoxy-N-[2-(4-morpholinyl)ethyl]-1,2-benzisoxazole-3-amine; 7-Bromo-6-methoxy-N-[2-(4-morpholinyl)ethyl]-1,2-benzisoxazol-3-amine; 5-Bromo-6-methoxy-N-[2-(4-morpholinyl)ethyl]-1,2-benzisoxazol-3-amine; 3-[[2-(4-Morpholinyl)ethyl]amino]-1,2-benzisoxazol-6-yl dimethylcarbamate; 3-[[(Methylamino)carbonyl][2-(4-morpholinyl)ethyl]amino]-1,2-benzisoxazolo-6-yl methylcarbamate; 3-[[(Methylamino)carbonyl][2-(4-morpholinyl)ethyl]-amino]-1,2benzisoxazol-5-yl methylcarbamate; 6-Methoxymethoxy-N-[2-(4-thiomorpholinyl)ethyl]-1,2-benzisoxazol-3-amine; 3-[[2-(4-Thiomorpholinyl)ethyl]amino]-1,2-benzisoxazol-6-ol; 6-Methoxy-N-methyl-N-[2-[4-(1-phenylmethyl)piperdinyl]-1,2-benzisoxazol-3-amine; 7-Bromo-3-[N-methyl, N-2-(4-morpholinyl)ethyl]amino-1,2-benzisoxazol-6-ol; 7-Bromo-3-[N-methyl,N-2-(4-morpholinyl)ethyl]amino-1,2-benzisoxazol-6-yl-dimethylcarbamate, and pharmaceutically acceptable salts thereof.

Examples of the preparation of these compounds and pharmaceutical compositions comprising the compounds are disclosed in e.g., U.S. Pat. No. 5,486,541 (issued Jan. 23, 1996); U.S. Pat. No. 5,130,327 (issued Jul. 14, 1992); U.S. Pat. No. 5,169,868 (issued Dec. 8, 1992); U.S. Pat. No. 5,326,770 (issued Jul. 5, 1994); U.S. Pat. No. 5,380,755 (issued Jan. 10, 1995); U.S. Pat. No. 5,811,456 (issued Sep. 22, 1998); 5,525,619 (issued Jun. 11, 1996); U.S. Pat. No. 6,060,516 (issued May 9, 2000); U.S. Pat. No. 7,235,581 (issued Jun. 26, 2007); U.S. Pat. No. 7,173,023 (issued Feb. 6, 2007); U.S. Pat. No. 7,087,612 (issued Aug. 8, 2006); U.S. Pat. No. 7,053,245 (issued May 30, 2006); U.S. Pat. No. 6,762,320 (issued Jul. 13, 2004); U.S. Pat. No. 6,900,354 (issued May 31, 2005); U.S. Pat. No. 6,951,884 (issued Oct. 4, 2005); U.S. Pat. No. 6,846,832 (issued Jan. 25, 2005); U.S. Pat. No. 4,650,907 (issued Mar. 17, 1987); U.S. Pat. No. 4,699,928 (issued Oct. 13, 1987); U.S. Pat. No. 5,380,861 (issued Jan. 10, 1995); U.S. Pat. No. 5,384,312 (Jan. 24, 1995); U.S. Pat. No. 5,529,988 (Jun. 25, 1996); U.S. Pat. No. 5,532,397 (Jul. 2, 1996); U.S. Pat. No. 6,660,736 (issued Dec. 9, 2003); U.S. Pat. No. 6,818,774 (issued Nov. 16, 2004); U.S. Pat. No. 6,667,327 (issued Dec. 23, 2003); U.S. Pat. No. 4,470,993 (issued Sep. 11, 1984); U.S. Pat. No. 5,641,785 (issued Jun. 24, 1997); U.S. Pat. No. 4,262,004 (issued Apr. 14, 1981); U.S. Pat. No. 4,346,102 (issued Aug. 24, 1982); U.S. Pat. No. 4,600,719 (issued Jul. 15, 1986); U.S. Pat. No. 4,471,117 (issued Sep. 11, 1984); U.S. Pat. No. 5,356,916 (issued Oct. 18, 1994); U.S. Pat. No. 5,494,908 (issued Feb. 27, 1996); U.S. Pat. No. 5,475,014 (issued Dec. 12, 1995); U.S. Pat. No. 5,238,962 (issued Aug. 24, 1993); U.S. Pat. No. 5,380,755 (issued Jan. 10, 1995); U.S. Pat. No. 5,298,626 (issued Mar. 29, 1994); U.S. Pat. No. 4,210,655 (issued Jul. 1, 1980); U.S. Pat. No. 4,042,584 (issued Aug. 16, 1977); U.S. Pat. No. 5,100,891 (issued Mar. 31, 1992); U.S. Pat. No. 4,822,812 (issued Apr. 18, 1989); U.S. Pat. No. 4,764,522 (issued Aug. 16, 1988); U.S. Pat. No. 4,705,796 (issued Nov. 10, 1987); and U.S. Pat. No. 4,616,032 (Oct. 7, 1986), each of which is hereby incorporated by referenced in their entireties.

In one embodiment of the invention, the compound useful for increasing UCHL1 activity (e.g., mRNA, protein levels, and/or hydrolase activity) is a polyamine, or an analog or derivative thereof. Examples of such compounds include, but are not limited to, alkylpolyaminoguanidines, alkylpolyaminobiguanides, tetramines, and pentamines and pharmaceutically acceptable salts thereof. In specific aspects of this embodiment, the polyamine is a guanidine or biguanides analog or derivative.

In one embodiment of the invention, the polyamine compound (or analog or derivative thereof) useful for increasing UCHL1 activity (e.g., mRNA, protein levels, and/or hydrolase activity) is of the formula E-NH—B-A-B—NH—B-A-B—NH—B-A-B—NH—B-A-B—NH-E. According to this embodiment, A is independently chosen from the group consisting of C1-C6 alkyl, C2-C6 alkenyl, C2-C6 alkynyl, C3-C6 cycloalkyl, C3-C6 aryl, and C3-C6 cycloalkenyl; B is independently chosen from the group consistingof a single bond, C1-C6 alkyl, and C2-C6 alkenyl; E is independently selected from the group consisting of H, C1-C6 alkyl, C2-C6 alkenyl, C2-C6 alkynyl, C3-C6 cycloalkyl, C3-C6 aryl, C3-C6 cycloalkenyl; with the proviso that either at least one A moiety is chosen from the group consisting of C2-C6 alkenyl, C2-C6 alkynyl, C3-C6 cycloalkyl, C3-C6 aryl, and C3-C6 cycloalkenyl, or at least one B moiety is chosen from the group consisting of C2-C6 alkenyl; and any salt or stereoisomer thereof.

In one embodiment of the invention, the polyamine compound (or analog or derivative thereof) useful for increasing UCHL1 activity (e.g., mRNA, protein levels, and/or hydrolase activity) is of the formula E-NH—B-A-B—NH—B-A-B—NH—B-A-B—NH(—B-A-B—NH)x-E. According to this embodiment, A is independently chosen from the group consisting of C1-C6 alkyl, C2-C6 alkenyl, C2-C6 alkynyl, C3-C6 cycloalkyl, C3-C6 aryl, and C3-C6 cycloalkenyl; B is independently chosen from the group consisting of a single bond, C1-C6 alkyl, and C2-C6 alkenyl; E is independently chosen from the group consisting of H, C1-C6 alkyl, C2-C6 alkenyl, C2-C6 alkynyl, C3-C6 cycloalkyl, C3-C6 aryl, and C3-C6 cycloalkenyl; and x is an integer from 2 to 16; with the proviso that either at least one A moiety is chosen from the group consisting of C2-C6 alkenyl, C2-C6 alkynyl, C3-C6 cycloalkyl, C3-C6 aryl, and C3-C6 cycloalkenyl, or at least one B moiety is selected from the group consisting of C2-C6 alkenyl; and any salt or H stereoisomer thereof.

Specific polyamine compounds include 1,11-bis{N²,N³-dimethyl-N¹-guanidino}-4,8-diazaundecane; 1,15-bis{N⁵-[3,3-(diphenyl)propyl]-N¹-biguanido}-4,12-diazapentadecane; BENSpm; N¹,N¹¹-bis(ethyl)norspermine; CPENSpm, N¹-ethyl-N¹¹-[(cyclopropyl)methyl]-4,8,-diazaundecane; CHENSpm; N¹-ethyl-N¹¹-[cyclohepthyl)methyl]-4,8,-diazaundecane; MTT, 3-(4,5-dimethylthiazol-2-yl)-2,5-diphenyltetrazolium bromide.

Examples of the synthesis of such polyamine compounds are known in the art and given in e.g., EP 1 177 197 B1 (WO 2000/066587), Bacchi et al. Antimicrobial Agents and Chemotherapy, January 2002, p. 55-61, Vol. 46, No. 1, Huang et al. Clinical Cancer Research Vol. 9, 2769-2777, July 2003, each of which is hereby incorporated by reference in its entirety.

Inhibitors of the function of AOF2 include Spermine; N-Acetyl-D-Glucosamine; Md172527 (N,N′-bis (2,3-butadienyl)-1,4-butane-diamine); alpha-D-mannose; alpha-D-fucose; Flavin-Adenine Dinucleotide; octane 1,8-diamine; L-deprenyl and tranylcypromine.

Demethylation agents such as 5-azacytidine and 5-aza-2′-deoxycytidine may also be useful in the method of the invention.

Any of these small molecule chemical agents would be useful in the treatment or prevention of neurodegenerative diseases, and particularly in the treatment of Lewy body disorders.

Further, a cell based screen for molecules that inhibit the UCHL1 inhibitory complex may be done by using a cell line expressing REST with low/no levels of UCHL1 in which a reporter cassette (e.g. GFP or luciferin) was inserted in its original genomic environment or in which a reporter construct fusing the UCHL1 regulatory domains with a reporter cassette was inserted randomly into the genome; and the screening for reactivation and expression of the reporter gene. In this way; inhibitors directed against additional components of the repressor complex can be discovered. We would expect such inhibitors to be useful in the treatment or prevention of neurodegenerative diseases, and particularly in the treatment of Lewy body disorders. Such inhibitors may include compounds, extracts or biological molecules, such as siRNA, miRNA, antibodies, and proteins.

In one embodiment, the invention provides a method of treating a patient by administering a combination of two or more inhibitors; for example the first may inhibit one protein belonging to the transcriptional repressor complex from the UCHL1 gene such as REST, sin3a, HDAC1,HDAC2, MeCP2, AOF2, RCOR1, JARID1C, BAF57, BAF170 and BRG1, and the second may inhibit a different member of the aforementioned complex. Alternatively or additionally the inhibitor may indirectly inhibit the complex by altering the transcription, translation, subcellular localisation or activity of one or several components of the complex. Any combination of inhibitors may be used and is not restricted to the example above.

In one embodiment, the invention uses an RNAi approach to directly or indirectly inhibit proteins in the transcriptional repressor complex, as would be apparent to the person skilled in the art.

An alternative approach uses microRNAs (miRNAs; miRs). miRs are small double stranded RNA molecules that are encoded in miRNA precursor genes. miRNA precursors mRNAs are transcribed; fold and are processed by the proteins Drosha and DICER to 20-25 base pair double-stranded RNA molecules. miRs negatively regulate expression of their target genes at the posttranscriptional level. In recent publications; computer algorithms were used to identify REST/NRSF target sequences (UCHL1 was not among the targets identified). Wu and Xi (2006) have used these algorithms to identify a set of miRNAs (hsa-miR-124a, hsa-miR-132, hsa-miR-135a, hsa-miR-153, hsa-miR-218, hsa-miR-29b, hsa-miR-9, hsa-miR-9*) whose expression may be regulated by the REST complex. The targets of the identified miRNAs, on the other hand, are the different components of the REST complex (a regulation feedback loop). Given that according to this invention REST was shown to regulate the UCHL1 promoter, the exogenous application of miRs that target the components of the REST complex could, (alone or in a mix containing at least one but possibly preferably several of these miRNAs), be an efficient way to release UCHL1 expression which would not be subject to the negative feedback loop existing in the human body. Any of the miRNAs listed above (available from Ambion) are suitable for use according to the method of the invention.

Yet another alternative approach uses small double stranded interference RNAs (siRNAs) directed against the members of the complex regulating the UCHL1 promoter. These can directly or indirectly inhibit the repression complex and release the control over the expression of UCHL1. A similar effect can be achieved with or short hairpin RNAs (shRNAs).

Examples of suitable siRNAs are listed in the table below:

| | | | | Ambion ID |
|---|---|---|---|---|
| HDAC1 | human | HPLC Purified, 5 nmol, | Validated | ID # 120418 |
| | | Annealed, w/out sequence | siRNA | (NM_004964) |
| HDAC2 | human | HPLC Purified, 5 nmol, | Validated | ID # 120210 |
| | | Annealed, w/out sequence | siRNA | (NM_001527) |
| HDAC6 | human | HPLC Purified, 5 nmol, | Validated | ID # 120452 |
| | | Annealed, w/out sequence | siRNA | (NM_006044) |
| AOF2 | human | HPLC Purified, 5 nmol, | Validated | ID # 118783 |
| | | Annealed, w/out sequence | siRNA | (NM_001009999, |
| | | | | NM_015013) |
| REST | human | Standard, 5 nmol, Annealed | Pre-designed | ID # 115696 |
| | | | siRNA | (NM_005612) |
| MECP2 | human | Standard, 5 nmol, Annealed | Pre-designed | ID # 143938 |
| | | | siRNA | (NM_004992) |
| SIN3A | human | Standard, 5 nmol, Annealed | Pre-designed | ID # 108733 |
| | | | siRNA | (NM_015477) |
| RCOR1 | human | Standard, 5 nmol, Annealed | Pre-designed | ID #136796; |
| | | | siRNA1) | (NM_015156) |
| JARID1C | human | Standard, 5 nmol, Annealed | Pre-designed | ID #115486; |
| | | | siRNA | (NM_004187) |

A further approach is to use antisense technology to inhibit proteins in the transcriptional repressor complex, as would be apparent to the person skilled in the art.

Another approach is the use of monoclonal antibodies directed against proteins in the transcriptional repressor complex. As the complex exerts its activity in the nucleus, the antibodies employed are preferably designed to be able to target and act in the cell nucleus (e.g. by fusion to the monoclonal antibody 3E10 Fv fragment (Hansen JE et al. Antibody-mediated p53 protein therapy prevents liver metastasis in vivo. Cancer Res. 2007 Feb. 15; 67(4):1769-74). Alternatively the antibodies may be designed to sequester proteins of the complex in the cytosol. Monoclonal antibodies have been described in the literature that would be suitable for use according to the present invention (e.g. Battaglioni et al. REST repression of neuronal genes requires components of the hSWI.SNF complex. J Biol Chem. 2002 Oct. 25; 277(43):41038-45). Antibodies directed against the members of the complex regulating the UCHL1 promoter can be generated by standard methods involving the fusion of antibody secreting B cells with cell lines selected for their ability to confer in vitro immortality on the antibody secreting cells. Alternatively, DNA encoding monoclonal antibodies, antigen binding chains or domains can be cloned and expressed using standard methods of recombinant DNA technology. Recombinant antigen binding molecules can be manipulated to improve therapeutic properties such as specificity, affinity, half-life and lack of immunogenicity.

Rodent (e.g. rat or mouse) or other non-human animal (e.g. horse) antibodies can be used according to the invention. However, for use in man it is preferred that the antibody has been engineered to limit the anti-globulin response. Examples of antibodies engineered in this way are chimeric antibodies (where the constant regions of a non-human antibody are replaced by human constant regions) and humanised antibodies where the antibody is engineered to appear human to the immune system of the recipient. Examples of humanised antibodies are CDR-grafted antibodies where as well as replacing the constant regions of a non-human antibody with a human constant region, the framework regions of the variable regions are also replaced by human variable regions.

The agent that represses the transcriptional complex that represses the promoter of the UCHL1 gene may be administered to the patient in a number of ways. For example use could be made of liposomes, nanoparticles, viral vectors, and the like. In particular it is preferred to use a delivery method that will allow the agent to cross the blood-brain barrier.

The products and methods of the invention will now be illustrated by the following examples which are not intended to limiting. The scope of the invention is defined by the appended claims.

### Example 1

### Weak Potential NRSF/REST Binding Sites are Present in the UCHL1 Regulatory Region

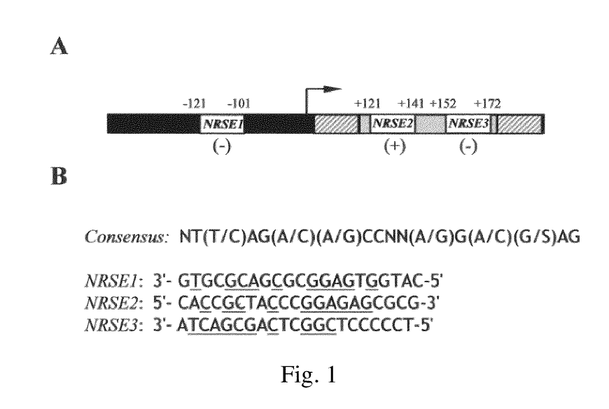

As a result of the previous findings showing that methylation of the UCHL1 gene promoter was not responsible for reduced UCHL1 levels in LBDs, an in silico analysis of UCHL1 gene promoter sequence was performed using the MatInspector software (Cartharius et al., 2005, Bioinformatics 21, 2933-2942). The analysis predicted three potential functional neuron-restrictive silencer elements (NRSE) which are the binding site for the neuronal restrictive silencer factor (NRSF/REST). NRSE1 (0.69) was located in the complementary DNA chain, upstream from the transcription start site between positions −121 and −101 by (FIG. 1A). NRSE2 (0.71) and NRSE3 (0.67) were located in the intron 1, in the coding and complementary chains, respectively. Both NRSE elements were located very close together, being separated only by 11 bp. The sequence of all NRSE elements and the consensus sequence are shown in FIG. 1B.

### Example 2

### NRSF/REST is a Putative Regulator of UCHL1, and its Expression Level is Inversely Related to UCHL1 in the Frontal Cortex in PD and DLB

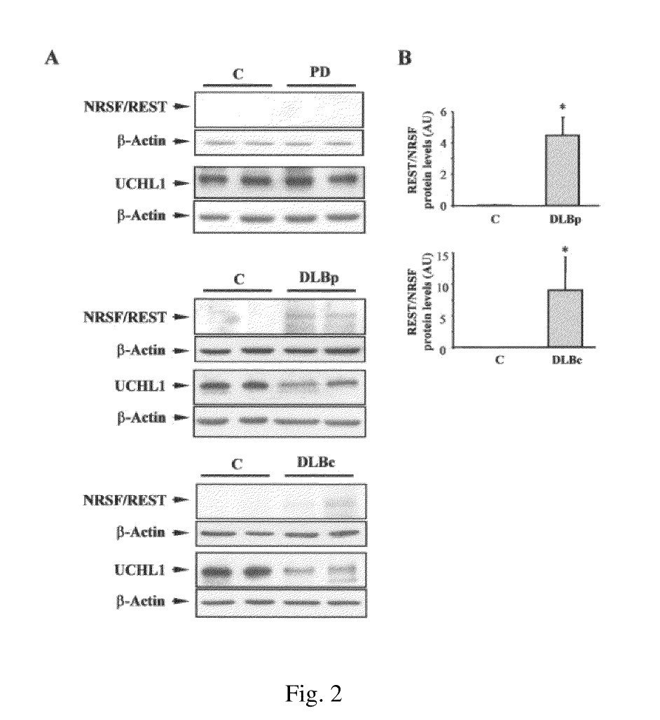

We recently showed that UCHL1 protein levels are reduced in the cerebral cortex in DLB but remain unchanged in the cerebral cortex of PD samples (Barrachina et al., 2006, Neurobiol. Dis. 22, 265-273). As NRSF is a repressor transcription factor, we tested whether there was an inverse relationship between UCHL1 and NRSF protein levels, as determined by Western blotting, in the frontal cortex in PD, DLBp, DLBc and agematched controls. NRSF levels were not detected in control and PD samples but were increased in DLBp and DLBc samples (FIG. 2). Moreover, increased NRSF protein levels occurred, in the cerebral cortex, in parallel with reduced UCHL1 protein levels (Barrachina et al., 2006).

### Example 3

### NRSF and UCHL1 Expression Levels are Inversely Related in Cell Lines

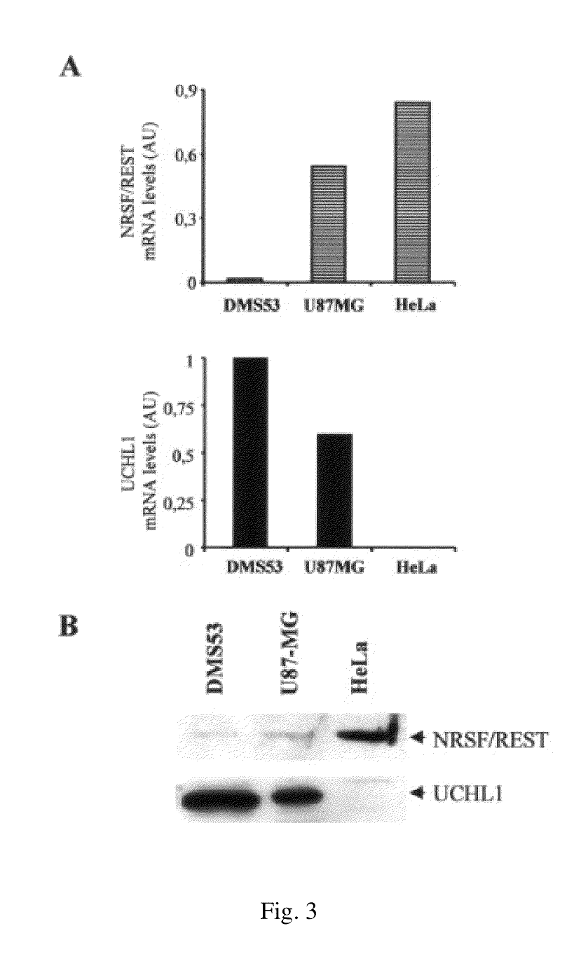

To verify the functional relationship between NRSF and UCHL1, we used a human lung carcinoma cell line (DMS53, small cell lung cancer), human glioblastoma cell line (U87-MG) and human cervical cancer cell line (HeLa). NRSF mRNA levels were absent in DMS53 cells but higher in U87-MG and, particularly, in HeLa cells (FIG. 3A). In contrast, UCHL1 mRNA levels were very high in NRSF-negative DMS53 cells, lower in U87-MG and undetectable in NRSF-positive HeLa cells. The same situation was found in relation to NRSF and UCHL1 protein (FIG. 3B).

### Example 4

### Exogenous Expression of REST/NRSF Reduces UCHL1 Expression

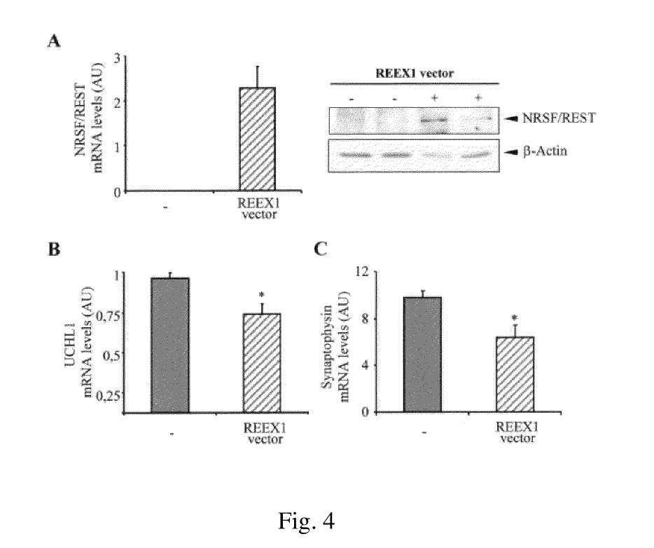

We then tested whether exogenous expression of NRSF attenuated endogenous UCHL1 mRNA levels in cells with very low levels of NRSF. The REEX1 vector, coding for human full-length NRSF cDNA, was transiently transfected in DMS53 cells, thereby increasing NRSF mRNA and protein levels (FIG. 4A). This over-expression reduced by 27% the expression levels of UCHL1 mRNA in the same cells (p<0.01, ANOVA with post-hoc LSD test) (FIG. 4B). As a positive control, we detected that NRSF over-expression reduced by 34% the expression levels of synaptophysin mRNA (p<0.01, ANOVA with post-hoc LSD test) (FIG. 4C), as previously described (Lietz et al., 2003).

### Example 5

### Inhibition of NRSF/REST Releases UCHL1 Repression

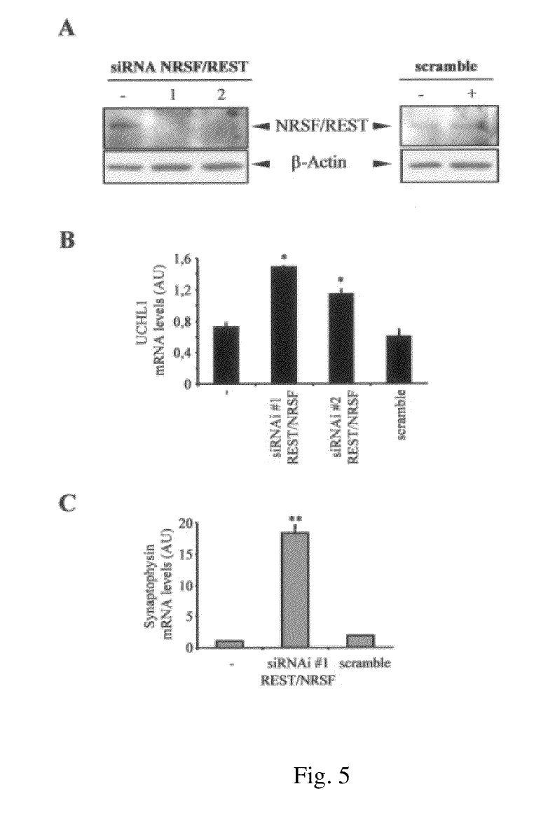

We also tested the effect of NRSF siRNA transfection in U87-MG cells. As shown in FIG. 5A, endogenous NRSF protein levels were reduced after transfection with NRSF siRNA#1 and siRNA#2, but remained unchanged with scramble siRNA. Reduction of NRSF expression was accompanied by increased expression of endogenous UCHL1 mRNA levels (p<0.01, ANOVA with post-hoc LSD test). No changes were found after transfection of scramble siRNA (FIG. 5B). Transfection of siRNA#1 upregulated endogenous expression of synaptophysin mRNA levels (p<0.001, ANOVA with post-hoc LSD test) (FIG. 5C).

### Example 6

### NRSF Interacts Directly with the UCHL1 Promoter

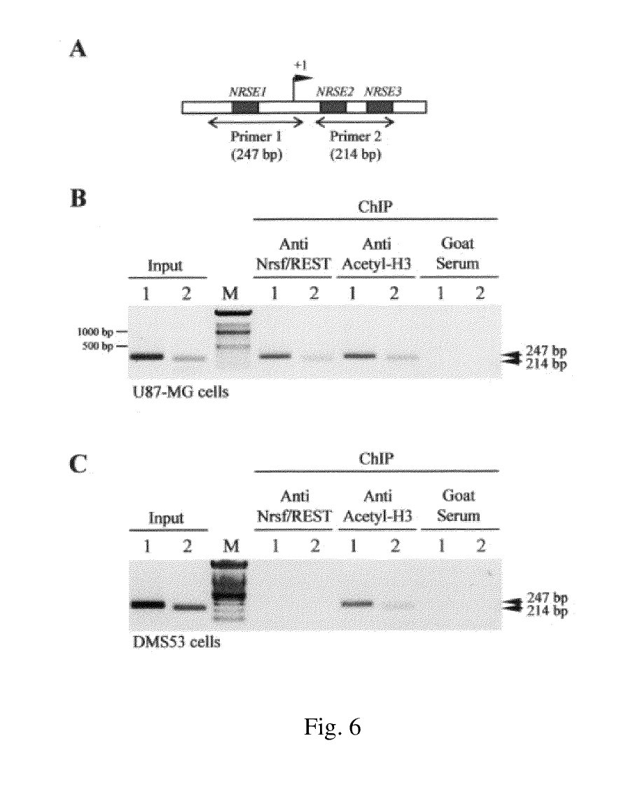

Having identified a functional relationship between NRSF and UCHL1, we proceeded to examine the interaction of NRSF with the UCHL1 gene promoter to further support the concept that NRSF regulates endogenous UCHL1 transcription by binding to its promoter. For this purpose, we carried out ChIP assays in U87-MG, HeLa and DMS53 cells. After the cross-linking of proteins and DNA with formaldehyde, sonicated cell lysates from each cell line were subjected to immunoprecipitation with the goat polyclonal anti-NRSF antibody. The precipitated DNA fragments were amplified with two sets of primers: set 1 spanned a 247 by region covering the NRSE1 and set 2 spanned a 214 by region covering the NRSE2 and NRSE3 of the UCHL1 gene promoter (FIG. 6A). In U87-MG and HeLa cells, ChIP PCR products were detected with the NRSF and acetyl-histone 3 antibodies but not with a goat serum used as a negative control (FIG. 6B). These results demonstrate that NRSF binds to the NRSE regions of UCHL1 gene promoter in both cell lines. By contrast, the same analysis performed in DMS53 cells revealed the absence of binding of NRSF to NRSE1 and the region covering NRSE2 and NRSE3 of UCHL1 gene promoter as no PCR amplification is obtained using both set of primers in the anti-NRSF DNA immunoprecipitated (FIG. 6C).

### Example 7

### Identification of NRSF/REST Repressor Complex Components Required for Negative Regulation of UCHL1

To identify other components of the NRSF/REST repressor complex that regulates UCHL1; we transfected the NRSF/REST expressing human glioblastoma cell line (U87-MG) and evaluated the effect of the transfection of siRNAs directed to candidate components of the NRSF/REST repressor complex binding to the UCHL1 promoter on UCHL1 mRNA expression by Taqman PCR.

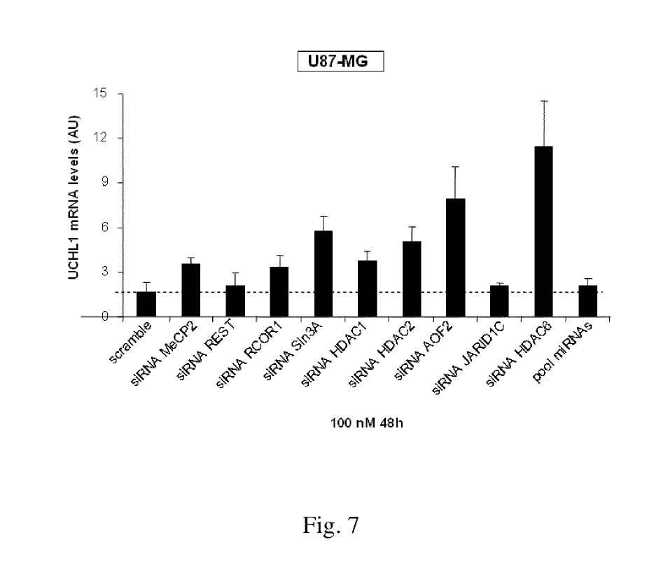

No increase of UCHL1 mRNA expression was observed in non transfected cells or in cells transfected at 100 nM concentration with siRNAs scrambled siRNA. A clear induction of expression was observed using siRNAs directed against MeCP2, RCOR1, sin3A, HDAC1,HDAC2, and AOF2 (direct inhibition of components the repression complex). In this experiment, lower inductions were observed with siRNAs directed against REST and JARID1C (FIG. 7).

### Example 8

### Identification of Factors NRSF/REST Repressor Complex Components Required for Negative Regulation of UCHL1

We also transfected the NRSF/REST expressing human glioblastoma cell line (U87-MG) with an siRNA directed against HDAC6.HDAC6 is not thought to participate directly in the regulatory complex that regulates the expression of the UCHL1 promoter. Nevertheless, a strong induction of UCHL1 mRNA expression was observed using an siRNA directed against HDAC6 at 100 nM concentration. (FIG. 7). Analysis of the role of HDAC6 in

### Example 9

### Inhibition of the REST/NRSF Complex and Expression of UCHL1 by Application of miRs Targeting Components of the REST/NRSF Complex

We transfected the NRSF/REST expressing human glioblastoma cell line (U87-MG) and evaluated the effect of the transfection of miRs targeting the NRSF/REST repressor complex on UCHL1 mRNA expression by Taqman PCR.

No increase of UCHL1 mRNA expression was observed in non transfected cells or in cells transfected at 100 nM concentration with scrambled siRNA. On the contrary, transfection with a miR mix composed of hsa-miR-124a, hsa-miR-132, hsa-miR-135a, hsa-miR-153, hsa-miR-218, hsa-miR-29b, hsa-miR-9, and hsa-miR-9* (100 nM total miRNA concentration) caused a small increase in the UCHL1 mRNA expression levels (FIG. 7).

### Example 10

### Application of HDAC Inhibitors Increases UCHL1 Expression Levels

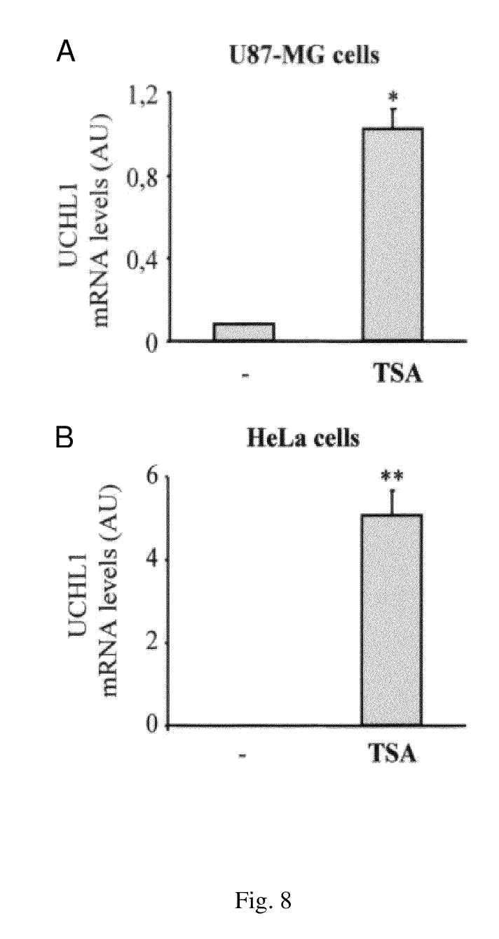

NRSF has been known to recruit histone deacetylases (HDAC) to act as repressors through chromatin remodelling (Naruse et al., 1999;Huang et al., 1999). To assess whether the repression of UCHL1 promoter activity is HDAC-dependent, we used TSA, a specific inhibitor of HDAC in U87-MG, HeLa and DMS53 cells. As shown in FIG. 8A, the inhibition of histone deacetylase activity by TSA 100 nM for 24 h was sufficient to increase UCHL1 mRNA levels in U87-MG cells (p<0.01, ANOVA with post-hoc LSD test) and to induce its expression in HeLa cells FIG. 8B with respect to non-treated cells (p<0.001, ANOVA with post-hoc LSD test). By contrast, the mRNA levels of endogenous UCHL1 in DMS53 cells remained unchanged after TSA treatment, a phenomenon compatible with the low NRSF protein levels detected in these cells. TSA treatment did not affect the endogenous NRSF mRNA levels in U87-MG and HeLa cells.

We treated the NRSF/REST expressing human glioblastoma cell line (U87-MG) during 72 h with 5 ₁AM HDAC inhibitors M344 (selective for HDAC6 over HDAC1), MOCPAC (selective for HDAc 1 over HDAC6) and BATCP (selective for HDAC6 over HDAC1), and evaluated the effect on UCHL1 mRNA expression by Taqman PCR.

No increase of UCHL1 mRNA expression was observed in non treated cells or cells treated with the vehicle. On the contrary, the treatment with HDAC inhibitors increased the UCHL1 mRNA expression levels.

### Example 11

### UCHL1 Promoter Methylation is Not Consistently Increased in Lewy Body Diseases

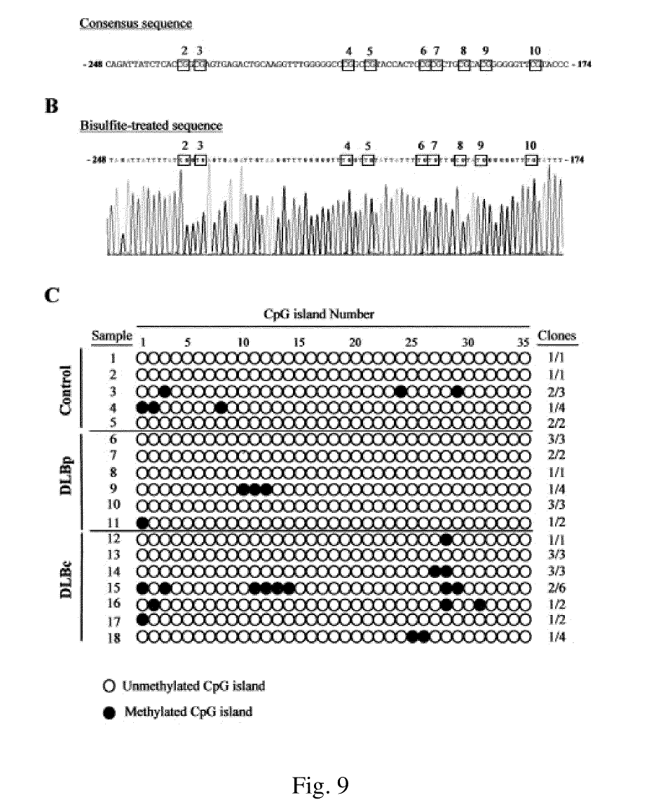

We analysed the methylation status of the minimal promoter region of the UCHL1 gene, spanning the transcription start site and exons 1 and 2 (GenBank accession n° X17377). For this purpose, genomic DNA from post-mortem cortical brain samples of patients with Dementia with Lewy Bodies pure form (DLBp) and common form (DLBc) and age-matched controls was purified and treated with bisulfite. This treatment allows the analysis of the methylation status at each cytosine in a CpG island. 5-methylcytosine remains non-reactive to bisulfite whereas non-methylated cytosines are replaced by thymine. All remaining cytosines represent, after sequence analysis, methylated cytosines (compare FIGS. 9A and B). We found that the vast majority of the 35 CpG islands described in the UCHL1 gene promoter were non-methylated in age-matched controls and in DLB samples (FIG. 9C). For example, the sample number 3 presented only three methylated positions (CpG islands 3, 24 and 29) in two of three clones analysed. Sample 4 presented three methylated positions in one of the four clones examined (CpG islands 1, 2 and 8). Sample 9 presented three methylated positions in one of the four clones analysed (positions 10-12). Only position 1 was methylated in one of the two analysed clones of sample 11. A slight increase in the number of methylated positions was found in the clone sequence from DLBc samples, although in no case did methylation sites exceed 25% of the 35 CpG islands analysed in the minimal UCHL1 gene promoter. Although we did not observe consistent methylation of the UCHL1 promoter in the frontal cortex of the analyzed DLB cases, it is known that transcriptional repression by REST can prime the DNA sequence for more stable repression by DNA methylation (Lunyak et al.. Corepressor-dependent silencing of chromosomal regions encoding neuronal genes. Science. 2002 Nov 29;298(5599):1747-52. Epub 24 October 2002)

### Example 12

### Application of Demethylating Agents Increases UCHL1 Expression in U87-MG and HeLa Cells

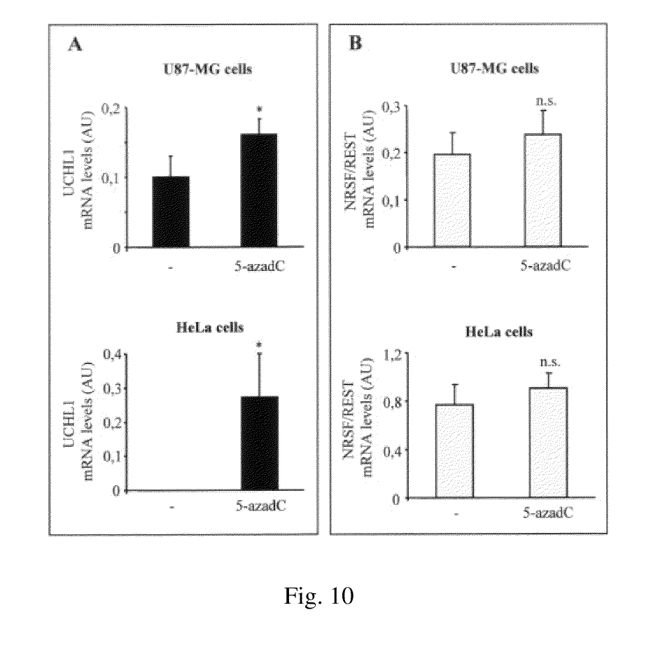

The proclivity for UCHL1 gene de-repression by a demethylating agent was also tested in U87-MG and HeLa cells. The treatment with 5-azacitidine 5 μM for 72 h up-regulated the expression of UCHL1 in U87-MG cells and induced its expression in HeLa cells (p<0.05, ANOVA with post-hoc LSD test) (FIG. 10A) without affecting the NRSF mRNA levels in either cell line (FIG. 10B). The induction was especially clear in HeLa cells; in which the UCHL1 promoter is known to be methylated.

### Example 13

### Cell-Based Screening Method for Molecules that Inhibit the UCHL1 Inhibitory Complex

Human reporter lines, for example, can be produced through plasmid or recombinant adeno associated viral vectors (rAAV) delivery of knock in constructs and homologous recombination with the endogenous UCHL1 gene; or by transient or stable transfection with promoter reporter fusion constructs.

### Targeting Constructs

Regions of homology at the UCHL1 locus can be amplified from genomic DNA obtained from U87-MG; HeLa cells or other cells in which UCHL1 expression is downregulated, using a High Fidelity DNA Polymerase (e.g. Pfu DNA Polymerase). Typically, 5 to 7 kb fragments are amplified from the upstream homology arm. For example, a DNA fragment covering 1.5 kb of the promoter of UCHL1, exon 1, intron 1, exon 2, intron 2, exon 3, intron 3 and exon 4 (a total of around 5 kb) can be amplified for the upstream homology region; which includes all the known regulatory elements for expression of the UCHL1 gene. While shorter fragments of 2 to 3 kb are sufficient for the downstream homology arm; for example the DNA fragment covering exon 5, intron 5, exon 6, intron 6 and exon 7 of the UCHL1 gene.

Unique restriction sites are embedded in the oligonucleotide primers used to amplify all fragments to facilitate cloning. Targeting plasmids are constructed by ligating the homology arms, and targeting/reporter cassette in the MCS of pBR322.

### Construction of the Targeting/Reporting Cassette

As an example, an adequate targeting/reporting cassette contains a hybrid 5′-regulatory element containing a short length of intron sequence followed by a splice acceptor site, an IRES, which permits the translation of the open reading frame (ORF) of the reporter gene (preferentially Luciferase; the synthetic firefly luc2 (Photinus pyralis) and Renilla hRluc (Renilla reniformis) included in the pGL4 vectors from Promega) from RNA transcripts initiating from upstream exons (Topaloglu et al. Nucleic Acids Res. 2005; 33(18): e158), followed by a polyadenylation site. Preferentially, the components of the targeting/reporter cassette have been codon optimized and engineered to reduce the number of consensus transcription factor binding sites to reduce the risk of anomalous transcription.

Integration of this cassette in the UCHL1 gene is useful to assess the effect of treatments on the transcriptional control of the UCHL1 gene. Alternative; the reporter gene may be translationally fused to part of the UCHL1 protein coding sequence. Integration of this cassette in the UCHL1 gene is useful to assess the effect of treatments on the transcriptional+translational control of the UCHL1 gene. Finally, the targeting/reporting cassette contains resistance gene (e.g. neo) expressed from its own promoter and provided with its own polyadenylation site for selection of stably transformed cells.

rAAV targeting constructs are assembled by ligation of homology arms and selectable marker cassettes, amplified using a high fidelity DNA Polymerase (e.g. DNA polymerase) from the targeting plasmid vector using oligonucleotide primers with embedded unique restriction sites allow inserted between the two NotI sites of pAAV-MCS, an AAV shuttle vector that carries the two inverted terminal repeat (ITR) sequences necessary for viral packaging (Stratagene). Typically, homology arms in rAAV targeting constructs need not be as long those for plasmid targeting vectors, 1 kb being sufficient.

Packaging of rAAV Targeting/Reporting Constructs

Infectious rAAV stocks can be produced with the AAV Helper-Free System (Stratagene) according to the manufacturer's protocols. Briefly, ITR-containing targeting constructs are co-transfected with the plasmids pAAV-RC and pHELPER. Approximately 5×106 AAV-293 cells are transfected with a mixture of 2.5 μg of each of the above three plasmids, using 54 μl of Lipofectamine (Invitrogen) as described by the manufacturer. Two days after transfection, cells are scraped into 1 ml of phosphate-buffered saline and frozen and thawed three times. The crude lysate is clarified by centrifugation.

Gene Targeting and Isolation of Recombinant Cell Lines Using rAAVs

Cells are grown in 25 cm2 flasks and infected with rAAV when ˜75% confluent. At the time of infection, medium was aspirated and 4 ml of medium containing rAAV lysate (0.5-2.5×105 viral particles) is added to each flask. Cells are washed with Hanks buffered saline solution and detached with trypsin (Invitrogen), 24 h after infection. Cells are replated in eight 96-well plates in medium containing geneticin (Invitrogen) at a final concentration of 0.4 mg/ml. Drug resistant colonies are grown for 3-4 weeks.

### Gene Targeting and Isolation of Recombinant Cell Lines Using Targeting/Reporter Plasmids

Approximately 5×106 cells are transfected with a mixture of 2.5 μg of the targeting/reporter plasmid, using 54 μl of Lipofectamine (Invitrogen) as described by the manufacturer. Cells are replated in eight 96-well plates in medium containing geneticin (Invitrogen) at a final concentration of 0.4 mg/ml. Drug resistant colonies are grown for 3-4 weeks.

### Analysis of Drug Resistant Colonies

Locus-specific integration of the targeting/reporting constructs (either plasmid or rAAV based) is assessed by PCR using primers outside the homology arms in combination with targeting cassette specific primers. In all cases, DNA polymerases fit for long PCR reactions (e.g. Pfu DNA polymerase) are employed.

### Promoter Fusions

Alternatively, the UCHL1 regulatory sequences, including the promoter sequences and the first intron of the UCHL1 gene, inserted in a pGL4 vector (Promega), transfected in transient or stable manner.

### Analysis of Reporter Gene Expression Levels

After generation of the reporter cell lines, the low expression level of the reporter gene in the cell lines and its induction by the application of the agents previously identified to effectively induce UCHL1 expression are verified, and cell lines with the correct response are selected.

Luciferase activity is assayed as described by the manufacturer (Promega). Sufficient Glo Lysis Buffer, equilibrated at 22° C., is added to the cells, equilibrated to room temperature; and incubated for 5 minutes at room temperature to allow lysis to occur. The lysates are transferred to luminometer tubes or plates and a volume of Bright Glo™ Assay Reagent equal of Glo Lysis Buffer is added and luminescence is measured with a luminometer.

Screening for Agents that Induce UCHL1 Expression

A cell line with correct expression response selected above is then employed to perform high throughput evaluation of compounds, extracts or biologicals (siRNA, miRNA, antibodies, and proteins) to assess their effectiveness in inducing UCHL1 expression. Assays can be performed either in plate format or using reverse transfected/treated cell arrays.

### Experimental Procedures

### Brain Samples

PD and DLB are considered α-synucleinopathies because abnormal α-synuclein is aggregated into Lewy bodies (LBs) and Lewy neurites in selected nuclei of the brain stem, spinal cord and autonomic ganglia. In addition, DLB is characterized by the widespread distribution of LBs and Lewy neurites in the cerebral cortex (Forno, 1996; Ince et al., 1998; Spillantini et al., 1998; Ince and McKeith, 2003; Jellinger and Mizuno, 2003). DLB is often accompanied by Alzheimer's disease (AD); this is considered the common form (DLBc). The pure form of DLB (DLBp) is characterized by minimal aA4-amyloid deposits and no tau pathology (Kosaka, 1993). The brains of six patients with PD, six DLBp, seven DLBc, and five aged-matched controls were obtained at autopsy, following informed consent of the patients or their relatives and the approval of the local ethics committees. Cases with prolonged agonal state, pyrexia, hypoxia, seizures or coma were excluded from the present study. Age range was between 57 and 91 years (mean age 75 years), and the average time between death and tissue processing was 6 h (between 2 and 13 h). pH range was between 6 and 7.Half of the brain was immediately cut into coronal sections, 1 cm thick, frozen on dry ice and stored at −80° C. until use. For morphological examinations, the brains were fixed by immersion in 10% buffered formalin for 2 or 3 weeks. Neuropathological characterization of PD was according to well-established neuropathological criteria (Jellinger and Mizuno, 2003).

Neuropathological characterization of DLB was according to consensus guidelines of the consortium on DLB international workshop (McKeith et al., 1996, 2000). Associated AD stages were further established depending on the amyloid deposition burden and neurofibrillary pathology, following the nomenclature of Braak and Braak (Braak and Braak, 1999). Stages of amyloid deposition refer to initial deposits in the basal neocortex (stage A), deposits extended to the association areas of the neocortex (stage B), and heavy deposition throughout the entire cortex (stage C). Stages of neurofibrillary pathology correspond to transentorhinal (I-II), limbic (III-IV) and neocortical (V and VI).

To further refine Alpha-synuclein pathology, staging of brain pathology related to sporadic PD proposed by Braak et al. (Braak et al., 2003) was used in the present study. Basically, stages 1 and 2 affect the medulla oblongata plus the pontine tegmentum; stage 3, the midbrain; stage 4, the basal prosencephalon and mesocortex; and stages 5 and 6, the neocortex. Clinically, all cases of PD had suffered from classical PD lasting from 8 to 15 years, and none of them had cognitive impairment.

Cases with DLB fulfilled the clinical criteria proposed by the consortium on DLB international workshop (McKeith et al., 1996, 2000). Control cases were considered in the absence of neurological symptoms and signs, and no abnormalities in the neuropathological study. The main neuropathological data in the present series are summarized in Table I. Biochemical studies were carried out in frozen samples of the frontal cortex (area 8). Control and diseased brains were processed in parallel.

Brain samples were obtained from the Institute of Neuropathology and University of Barcelona Brain Banks following the guidelines and approval of the local ethics committees.

### Bisulfite Treatment and PCR Amplification of Bisulfite-Treated DNA

1.5 μg of genomic DNA isolated from human frozen brain homogenate was re-suspended in 50 μl of water and denatured, adding 5.7 μl of 3M NaOH for 10 min at 37° C. Then 33 of 20 mM hydroquinone (Sigma) and 530 of 4.3 M sodium bisulfite (Sigma) at pH 5.0 were added. The DNA solution was incubated for 16 h at 50° C. After that, DNA samples were desalted through a column (Wizard DNA Clean-Up System, Promega) and eluted with 50 μl of water. Then, the eluted DNA was treated with 5.7 μl of 3M NaOH for 20 min at 37° C. Finally, DNA was precipitated, adding 1 μl of 10 mg/ml glycogen, 17 μl of 10M ammonium acetate and 450 μl of ethanol overnight at −80° C. The bisulfite-modified genomic DNA was re-suspended in 50 μl of water.

The conditions used in PCR amplification of bisulfite-modified genomic DNA have been previously described (Bittencourt-Rosas et al., 2001). The primers used were the CPGP9.5-Fow: 5′-TTAAAAggATTgTTTTATATATTTAAggAAT-3′ (SEQ ID NO:7) and CPGP9.5-Rev: 5′ -CACTCACTTTATTCAACATCTAAAAAAC-3′. (SEQ ID NO:8) The PCR product (473 bp) was cloned in TA pCRII vector (Invitrogen) and transformed in OneShot TOP10 chemically competent bacteria (Invitrogen). Several clones from each bisulfite-modified genomic DNA sample were sequenced using SP6 (5′-ATTTAggTgACACTATAg-3′) (SEQ ID NO:9) and T7 (5′-TAATACgACTCACTATAggg-3′) (SEQ ID NO:10) primers and the Big Dye Terminator v3.1 sequencing kit on an Abi Prism 3730 sequence detector (Applied Biosystems).

### Cell Culture

HeLa cells were maintained in Dulbecco's minimal essential medium (DMEM, Gibco, Invitrogen) supplemented with 10% foetal bovine serum. U87-MG cells (ATCC® number: HTB-14) were maintained in minimal essential medium (Eagle) with 2 mM L-glutamine and supplemented with 10% foetal bovine serum and 1 mM sodium pyruvate. DMS53 cells (ATCC® number: CRL-2062) were maintained in Waymouth's MB 752/1 medium (GIBCO) supplemented with 10% foetal bovine serum.

All cell lines were grown at 37° C. in a humidified atmosphere of 5% CO2. TSA was dissolved in ethanol and 5-azacytidine in water:acetic acid (1:1). For TSA treatment, cells were plated in 6-well dishes at a concentration of 105 cell/well and cultured overnight before activation. Cells were plated at a concentration of 50,000 cell/well for 5-azacytidine and also cultured overnight before treatment.

### Cell Transfection

DMS53 cells were plated in 6-well dishes at a concentration of 105 cells/well and cultured overnight before transfection. 1 μg of REEX1 vector (kindly provided by Dr. Gail Mandel) was transfected using lipofectamine™ 2000 (Invitrogen) following the instructions of the manufacturer. After 5 hours of post-transfection the medium was replaced by fresh medium. The efficiency of transfection was around 40% using the pEGFP-C1 vector (BD Biosciences Clontech).

siRNA and miR Transfection

U87-MG cells were plated in 6-well dishes at a concentration of 50,000 cells/well and cultured overnight before transfection. 100 nM of siRNA, a mix of miRs at lOnM each or scramble siRNA (Ambion, Cat. N° 4611) were transfected using lipofectamine™ 2000 (Invitrogen) following the instructions of the manufacturer. After 5 hours of post-transfection the medium was replaced by fresh medium. The analysis of the siRNA or miR transfection was performed 48 hours later.

All siRNAs and miRs used were from Ambion (Applied Biosystems): P NRSF/REST; 5′-GCUUAUUAUGCUGGCAAAUTT-3′; (SEQ ID NO:11)Ambion, Cat. N°16810, NRSF/REST; 5′-GCCUUCUAAUAAUGUGUCATT-3′; (SEQ ID NO:12) Ambion, Cat. N°16708,
HDAC1; ID #120418 (NM_{—}004964); AM51320
HDAC2; ID #120210 (NM_{—}001527); AM51320
HDAC6; ID #120452 (NM_{—}006044); AM51320
AOF2; ID #118783 (NM_{—}001009999, NM_{—}015013); AM51320
REST; ID #115696 (NM_{—}005612); AM16708
MECP2; ID #143938 (NM_{—}004992); AM16708
SIN3A; ID # 108733 (NM_{—}015477); AM16708
RCOR1; ID #136796; (NM_{—}015156); AM16708
JARID1C; ID #115486; (NM_{—}004187); AM1670
Silencer® Negative control #1 siRNA; AM4611
hsa-miR-124a; PM10245; AM17100
hsa-miR-132; PM10166; AM17100
hsa-miR-135a; PM11126; AM17100
hsa-miR-153; PM11007; AM17100
hsa-miR-218; PM10328; AM17100
hsa-miR-29b; PM10103; AM17100
hsa-miR-9; PM10022; AM17100
hsa-miR-9*; PM10156; AM17100

### Western Blot

Frozen frontal cortex (area 8; 100 mg) was directly homogenized in 1 ml lysis buffer (20 mM Hepes, 10 mM KCl, 1.5 mM MgCl2, 1 mM EDTA, 1 mM EGTA, 1 mM DDT, 2 mM PMSF, 1 μg/ml aprotinin, leupeptin and pepstatin) and then sonicated. Cell lines grown in 10 ml-plates were homogenized in the same way without sonication. Lysates were centrifuged at 2650 g for 10 minutes at 4° C., and protein concentration was determined with BCA (Pierce) method. 30 μg of total protein was boiled at 95° C. for 3 min and loaded in SDS-polyacrylamide gels with Tris-glycine running buffer.

Proteins were electrophoresed using a mini-protean system (Bio-Rad) and transferred to nitrocellulose membranes (Bio-Rad) with a Mini Trans-Blot electrophoresis transfer cell (Bio-Rad) for 1 h at 100 V.

Nitrocellulose membranes were blocked with Tween 20 TBS (TBST), containing 5% skimmed milk, for 30 min. Subsequently, the membranes were incubated at 4° C. overnight with one of the primary antibodies in TBST containing 3% BSA. The following antibodies were used: anti-REST (Abcam) used at a dilution of 1:250, anti-UCHL1 (AB5937, Chemicon) used at a dilution of 1:500, and anti-S-actin (clone AC-74, Sigma) diluted 1:10,000. After incubation with the primary antibody, the membranes were washed three times with TBST for 5 min at room temperature, and then incubated with the corresponding anti-rabbit, anti-goat or anti-mouse IgG antibody labelled with horseradish peroxidase (Dako) at a dilution of 1:1,000 (1:10,000 for S-actin) for 1 h at room temperature.

Subsequently, the membranes were washed five times, 5 min each, with

TBST at room temperature, and developed with the chemiluminescence ECL Western blotting system (Amersham/Pharmacia), followed by apposition of the membranes to autoradiographic films (Hyperfilm ECL, Amersham).

mRNA Isolation

The RNA from cell lines was purified with RNeasy Midi kit (Qiagen) following the protocol provided by the manufacturer. The concentration of each sample was obtained from A260 measurements. RNA integrity was tested using the Agilent 2100 BioAnalyzer (Agilent).

cDNA Synthesis

The retrotranscriptase reaction (100 ng RNA/μl) was performed using the High capacity cDNA Archive kit (Applied Biosystems) following the protocol provided by the supplier. Parallel reactions for each RNA sample were run in the absence of MultiScribe Reverse Transcriptase to assess the degree of contaminating genomic DNA.

### TaqMan PCR

The NRSF/REST TaqMan assay (Hs00194498_ml, TaqMan probe 5′-AGGAAGGCCGAATACAGTTATGGCC-3′) (SEQ ID NO:13) (Applied Biosystems) generates an amplicon of 79 by and is located at position 341 between 1 and 2 exon boundary of NM_{—}005612.3 transcript sequence.

The TaqMan assay for UCHL1 (Hs00188233_ml, TaqMan probe 5′-CCTGCTGAAGGACGCTGCCAAGGTC-3′) (SEQ ID NO:14) (Applied Biosystems) is located at position 648 between 8 and 9 exon boundary of NM_{—}004181.3 transcript sequence. It generates an amplicon of 100 bp. The TaqMan assay for Synaptophysin (Hs 00300531_ml, TaqMan probe 5′-CGAGTACCCCTTCAGGCTGCACCAA-3′) (SEQ ID NO:15) (Applied Biosystems), generates an amplicon of 63 by and is located at position 241 of NM_{—}003179.2 transcript sequence.

TaqMan PCR assays for NRSF/REST, UCHL1 and synaptophysin were performed in duplicate on cDNA samples in 96-well optical plates using an ABI Prism 7700 Sequence Detection system (Applied Biosystems). The plates were capped using optical caps (Applied Biosystems). For each 20 μl TaqMan reaction, 9 μl cDNA (diluted 1/50) was mixed with 1 μl 20× TaqMan® Gene Expression Assays and 10 μl of 2× TaqMan Universal PCR Master Mix (Applied Biosystems). Parallel assays for each sample were carried out with S-glucuronidase (GUSB) (Hs99999908_ml, TaqMan probe 5′-GACTGAACAGTCACCGACGAGAGTG-3′)(SEQ ID NO:16), for normalization. The reactions were carried out using the following parameters: 50° C. for 2 min, 95° C. for 10 min, and 40 cycles of 95° C. for 15 sec and 60° C. for 1 min. Standard curves were prepared for NRSF/REST, UCHL1, synaptohysin and GUSB using serial dilutions of cDNA from U87-MG cell line. Finally, all TaqMan PCR data were captured using the Sequence Detector Software (SDS version 1.9, Applied Biosystems).

The amount of targets (NRSF/REST, UCHL1 and synaptophysin) and endogenous reference (GUSB) was determined for each experimental sample from the appropriate standard curve, which was plotted showing the cycle threshold, Ct (y), versus log ng total control RNA (x). The amount of each target was divided by the endogenous reference amount to obtain a normalized target value (arbitrary units).

### Chromatin Immunoprecipitation (ChIP Assay)

ChIP assay was performed according to the manufacturer's protocol (Upstate) using 106 U87-MG, HeLa and DMS53 cells. 10 ₁..tg Anti-NRSF/REST (P-18×, sc-15118X Santa Cruz) and 10 μg antiacetylated H3 (residue Lys9, Cell Signalling) were used for immunoprecipitation. Purified DNA was resuspended in 20 μl of DNAse-free water and 1 μl was used as template in 25 μl of PCR reaction using GoTaq Flexi DNA Polymerase (Promega). Primer concentration was 200 nM. PCR primers were 5′-ACAAATCCCgTCTCCACAAC-3′(SEQ ID NO:17) and 5′-gCCTAgggAAgACgAAAAACA-3′ (SEQ ID NO:18) for the amplification of NRSE1 sequence of UCHL1 gene promoter. The NRSE2 and NRSE3 sequences were amplified with 5′-gCTCCgTAgCTgTTTTTCgT-3′ (SEQ ID NO:19) and 5′-gCCACTCACTTTgTTCAgCA-3′. (SEQ ID NO:20). The reaction was carried out using the following parameters: 95° C. for 2 min and 35 cycles of 95° C. for 30 sec, 65° C. for 30 sec and 72° C. for 30 sec. Finally, a last hold of 72° C. for 5 min was performed.

What is claimed is:

1. A method of treating or preventing a neurodegenerative disease in a patient suffering from such a condition which comprises identifying a patient in need of prevention or treatment and administering to said patient a therapeutically effective amount of an agent that represses the transcriptional complex that represses the promoter of the UCHL1 gene.

2. The method of claim 1 wherein said therapeutically effective amount of an agent is an amount of said agent sufficient to represses the transcriptional complex that represses the promoter of the UCHL1 gene.

3. The method of claim 2 wherein said therapeutically effective amount of an agent is an amount said agent sufficient to increase UCHL1 protein, mRNA or hydrolase activity.

4. The method of claim 2 wherein said patient in need of treatment or prevention is identified as having altered expression of REST.

5. The method according to claim 2 wherein the disease is Alzheimer's disease.

6. The method according to claim 2 wherein the disease is Alzheimer's disease.

7. The method according to claim 2, wherein the disease is a Lewy Body Disorder.

8. The method according to claim 2, wherein the disease is Huntington's Disease.

9. The method according to claim 1, wherein the agent is a small molecule that inhibits the function of one or more proteins selected from the group consisting of REST, sin3a, HDAC1,HDAC2, MeCP2, AOF2, RCOR1, JARID1C, BAF57, BAF170 and BRG1 in the transcriptional complex that represses the promoter of the UCHL1 gene.

10. The method according to claim 9, wherein the agent is a HDAC inhibitor.

11. The method according to claim 10, wherein the HDAC inhibitor is selected from Trichostatin A (TSA), Suberoylanilide hydroxamic acid (SAHA), N-Hydroxy-4-(Methyl{[5-(2-Pyridinyl)-2-Thienyl]Sulfonyl}Amino)Benzamide, 4-Dimethylamino-N-(6-Hydroxycarbamoyethyl)Benzamide-N-Hydroxy-7-(4-Dimethylaminobenzoyl)Aminoheptanamide, 7-[4-(Dimethylamino)Phenyll-N-Hydroxy-4,6-Dimethyl-7-Oxo-2,4-Heptadienamide, and Docosanol.

12. The method according to claim 10, wherein the HDAC inhibitor is a short-chain to medium-chain fatty acid or a derivative or analog thereof.

13. The method according to claim 9 wherein the agent is a small molecule that inhibits the function of AOF2

14. The method of claim 13 wherein said agent that is a small molecule that inhibits the function of AOF2 is selected from the group consisting of spermine; N-Acetyl-D-Glucosamine; Md172527 (N,N′-bis(2,3-butadienyl)-1,4-butane-diamine); alpha-D-mannose; alpha-D-fucose; Flavin-Adenine Dinucleotide; octane 1,8-diamine; L-deprenyl or tranylcypromine.

15. The method according to claim 1, wherein the inhibition is provided by administering a short hairpin RNA (shRNA), microRNA, antisense molecule, a small double stranded interference RNA (siRNA) for at least one of the genes that codes for a protein belonging to the transcriptional repressor complex from the UCHL1 gene, or for a gene that codes for a protein that affects the transcription, translation, subcellular localization or activity of one or several of the components of the transcriptional complex that represses the promoter of the UCHL1 gene.

16. The method according to claim 15, wherein the gene that codes for a protein belonging to the transcriptional repressor complex from the UCHL1 gene is selected from the group consisting of REST, sin3a, HDAC1,HDAC2, MeCP2, AOF2, RCOR1, JARID1C, BAF57, BAF170 and BRG1.

17. The method according to claim 1, wherein the inhibition is provided by administering a monoclonal antibody directed against at least one of the proteins belonging to the transcriptional repressor complex for the UCHL1 gene, or for a gene that codes for a protein that affects the transcription, translation, subcellular localization or activity of one or several of the components of the transcriptional complex that represses the promoter of the UCHL1 gene.

18. The method of claim 17 wherein said protein belonging to the transcriptional repressor complex from the UCHL1 gene is selected from REST, sin3a, HDAC1,HDAC2, MeCP2, AOF2, RCOR1, JARID1C, BAF57, BAF170 and BRG1.

19. A method for identifying a compound that represses the transcriptional complex that represses the expression of the UCHL1 promoter comprising providing a compound which is a small molecule inhibitor of a protein chosen from REST, sin3a, HDAC1,HDAC2, MeCP2, AOF2, RCOR1, JARID1C, BAF57, BAF170 and BRG1 and testing for the ability of said compound to repress the transcriptional complex that represses the promoter of the UCHL1 gene.

20. The method of claim 19 wherein said protein is AOF2.
